(19) United States
(12) Patent Application Publication
(10) Pub. No.: US 2025/0281598 A1
(43) Pub. Date: Sep. 11, 2025

(54) DNA DISPLAY OF FOLDED RNA LIBRARIES ENABLING RNA-SELEX WITHOUT REVERSE TRANSCRIPTION

(71) Applicant: Brandeis University, Waltham, MA (US)

(72) Inventors: Isaac J Krauss, Needham Heights, MA (US); Richard Redman, San Diego, CA (US)

(73) Assignee: Brandeis University, Waltham, MA (US)

(21) Appl. No.: 18/564,289

(22) PCT Filed: May 27, 2022
(86) PCT No.: PCT/US2022/031367

§ 371 Date: Nov. 27, 2023

Related U.S. Application Data
(60) Provisional application No. 63/194,002, filed on May 27, 2021.

(51) Int. Cl. A61K 39/21; A61K 39/00; A61K 39/385; C12N 15/10; C12N 15/115; C12Q 1/6804; C12Q 1/6811; G01N 33/569; G01N 33/68

(52) U.S. Cl. CPC A61K 39/21; A61K 39/385; C12N 15/1048; C12N 15/115; C12Q 1/6804; C12Q 1/6811; G01N 33/56988; G01N 33/6857; A61K 2039/53; G01N 2333/16

(57) ABSTRACT

The present application discloses a method for selecting an RNA molecule that binds to a target molecule that includes providing a pool of oligonucleotide complexes, exposing the pool to a target molecule and allowing the second region of the RNA to bind a target molecule; and selecting from the pool one or more oligonucleotide complexes comprising an RNA molecule having the second region bound to the target molecule. Further disclosed is an isolated RNA molecule, an immunogenic conjugate, a pharmaceutical composition, a method of inducing an immune response in an individual, a method of inhibiting HIV-1 infection or proliferation, and a method for detecting a neutralizing antibody in serum.

### CROSS REFERENCE TO RELATED APPLICATIONS

This application is a National Stage application of PCT/US2022/031367, filed May 27, 2022, which claims priority to U.S. Provisional Application No. 63/194,002, filed May 27, 2021, both of which are incorporated by reference in their entirety herein.

### STATEMENT REGARDING FEDERALLY SPONSORED RESEARCH & DEVELOPMENT

This invention was made with government support under grant numbers AI 090745 and AI 113737 awarded by the National Institutes of Health. The government has certain rights in this invention.

### SEQUENCE LISTING

The Instant Application contains a Sequence Listing which has been submitted electronically in ASCII format and is hereby incorporated by reference in its entirety. Said ASCII copy, created on May 27, 2022 is named “Replacement_Seq_List--107648.035” and is 10.3 KB (10,638 bytes) in size.

### FIELD

The present disclosure is directed to a method of selecting an RNA molecule that binds to a target molecule, including the selection of RNA molecules bearing one or more modifications such as modified bases and/or modified ribosyl-phosphate groups.

### BACKGROUND

First characterized in 1990 independently by the laboratories of Szostak and Gold, oligonucleotide aptamers are DNA or RNA with high affinity for proteins or small molecules (Ellington et al., Nature, 346:818-822 (1990); Tuerk et al., Science, 249:505-510 (1990); Gold et al., Cold Spring Harbor Perspect. Biol., 4 (2012); Stoltenburg et al., Biomol. Eng., 24:381-403 (2007)). Exhibiting advantages such as affinities comparable to those of antibodies, simplicity of synthesis, and general lack of immunogenicity, aptamers have found a place in the pharmaceutical market. The first aptamer drug approved by the FDA, Pegaptanib, targets age-related macular degeneration by binding to vascular endothelial growth factor (VEGF) (Kanwar et al., Curr. Med. Chem., 22:2539-2557 (2015); Keefe et al., Nat. Rev. Drug Discovery, 9:537-550 (2010); Sundaram et al., Eur. J. Pharm. Sci., 48:259-271 (2013); Sun et al., Mol. Ther.—Nucleic Acids, 3:e182 (2014); Santosh et al., BioMed Res. Int., 2014: 540451 (2014); Ruckman, et al., J. Biol. Chem., 273:20556-20567 (1998)). Several other aptamers are in late-stage clinical trials, and an unknown number are in laboratory development (Kanwar et al., Curr. Med. Chem., 22:2539-2557 (2015); Keefe et al., Nat. Rev. Drug Discovery, 9:537-550 (2010); Sundaram et al., Eur. J. Pharm. Sci., 48:259-271 (2013); Sun et al., Mol. Ther.—Nucleic Acids, 3:e182 (2014); Santosh et al., BioMed Res. Int., 2014: 540451 (2014)). Aptamers are typically discovered using standard molecular biology techniques through an in vitro selection process termed SELEX (Systematic Evolution of Ligands by EXponential enrichment) (Stoltenburg et al., Biomol. Eng., 24:381-403 (2007)). DNA libraries with random regions flanked by constant regions are synthesized using standard phosphoramidite chemistry and can be used directly in the selection of DNA aptamers. RNA libraries are generated by transcription of a T7 promoter-containing random DNA library using T7 RNA polymerase. Libraries are mixed with a target small molecule or protein and the bound fraction is recovered using a variety of isolation techniques. The recovered library is amplified with the polymerase chain reaction (PCR) for DNA aptamers or reverse transcription-PCR for RNA aptamers, the single-stranded DNA or RNA is regenerated from the PCR product and the selection cycle is repeated until the library is sufficiently enriched with target-binding aptamers.

Limitations to standard aptamer libraries (nuclease sensitivity, limited chemical diversity) have been overcome by the use of non-natural nucleotide analogs (Gold, et al., PLOS One, 5:e15004 (2010); Gupta et al., J. Biol. Chem., 289:8706-8719. (2014); Keefe et al., Curr. Opin. Chem. Biol., 12:448-456 (2008); Lapa et al., Mol. Biotechnol., 58:79-92 (2016); Ohsawa et al., Anal. Sci., 24:167-172 (2008); Shoji et al., J. Am. Chem. Soc., 129:1456-1464 (2007); Wang et al., Curr. Med. Chem., 18:4126-4138 (2011)). For instance, the incorporation of 2′-fluoro pyrimidines into RNA by T7 RNA polymerase variants results in significant serum nuclease resistance, a major requirement for drug development. To improve chemical diversity, base-modified DNA aptamers have been readily utilized, largely owing to the discovery that family B DNA polymerases (including Vent, KOD, and Pfu) can accommodate C5-substituted thymidine base analogs (Gold et al., Cold Spring Harbor Perspect. Biol., 4 (2012); Gold, et al., PLOS One, 5:e15004 (2010); Gupta et al., J. Biol. Chem., 289:8706-8719. (2014); Keefe et al., Curr. Opin. Chem. Biol., 12:448-456 (2008); Lapa et al., Mol. Biotechnol., 58:79-92 (2016); Ohsawa et al., Anal. Sci., 24:167-172 (2008); Kuwahara et al., Nucleic Acids Symp. Ser., 81-82 (2005); Kuwahara et al., Molecules, 15:5423-5444 (2010); Rohloff et al., Mol. Ther.—Nucleic Acids, 3:e201 (2014); M. Kuwahara et al., Nucleic Acids Res., 34:5383-5394 (2006); M. Kuwahara et al., Molecules, 15:8229 (2010)). In particular, SOMAmer technology, which incorporates short hydrophobic groups at the C5 position of uridine, improved the success rate of obtaining high-affinity aptamers from 30% to 80% for hundreds of targets (Rohloff et al., Mol. Ther.—Nucleic Acids, 3:e201 (2014)). Recently, the inventors reported a method termed SELection of Modified Aptamers, or SELMA, allowing for the incorporation of large modifications into DNA libraries, which was successfully used to obtain multivalent glycoclusters that mimic a conserved epitope on the HIV envelope protein gp120 (MacPherson et al., Angew. Chem., Int. Ed., 50:11238-11242 (2011); Temme et al., Chemistry, 19:17291-17295 (2013); Temme et al., Curr. Protoc. Chem. Biol., 7:73-92 (2015); Temme et al, J. Am. Chem. Soc., 136:1726-1729 (2014)).

However, compared to DNA, successes with base-modified RNA aptamer libraries have not been as wide-spread, despite the superior serum nuclease resistance of 2′-F-modified RNA and greater folding space sampling of RNA libraries. An obstacle to development of base-modified RNA-SELEX is that it would require that two different types of enzymes (RNA polymerase and reverse transcriptase) tolerate the modified bases. It would be desirable, therefore, to develop a modified SELEX procedure that facilitates the selection of RNA aptamers that may contain modified RNA analogues.

Many events in host-pathogen recognition, cell adhesion and cell signaling are mediated by carbohydrate-protein interactions (Glycan-Binding Proteins. In Essentials of Glycobiology, 3rd ed.; Varki, A. C., Richard D.; Esko, Jeffrey D., et al., Ed. Cold Spring Harbor Laboratory Press: Cold Spring Harbor (NY) (2015-2017)). Therefore, the design or discovery of carbohydrate structures that can faithfully mimic or interrupt interactions with carbohydrate binding proteins (CBPs) has potentially broad applications in medicine, including the development of vaccines (Heimburg-Molinaro et al., Vaccine, 29 (48): 8802-26 (2011); Astronomo et al., Nat. Rev. Drug Discov., 9 (4): 308-24 (2010); Horiya et al., Nat. Chem. Biol., 10 (12): 990-999 (2014); Bastida et al., Drug Discov. Today Technol., 35-36:45-56 (2020)) immunomodulatory agents (van Kooyk et al., Nat. Immunol., 9 (6): 593-601 (2008); Johnson et al., Trends Immunol. 34 (6): 290-298 (2013); Marth et al., Nat. Rev. Immunol. 8 (11): 874-887 (2008); Schnaar et al., J. Allergy Clin. Immunol., 135 (3): 609-615 (2015)), diagnostics (Hu et al., in Progress in Molecular Biology and Translational Science, Zhang, L., Ed. Academic Press: 162:1-24 (2019)), and anti-adhesion drugs against cancer and other diseases. Sharon et al., Biochim Biophys Acta, 1760 (4): 527-37 (2006) and Krishnamurthy et al., Nat. Commun. 6 (1): 6387 (2015) (c) Zhang et al., Blood, 136 (Supplement 1): 17-18 (2020).

Whereas CBPs typically have low affinity (K_{D}˜mM to μM) for individual glycan units, high affinity interactions are achieved through multivalent interactions with glycan clusters (Roy et al., Molecules, 21 (5): 629 (2016); Dam et al., In Advances in Carbohydrate Chemistry and Biochemistry, Horton, D., Ed. Academic Press 63:139-164 (2010); Wittmann et al. Chem. Soc. Rev., 42 (10): 4492-4503 (2013); Wittmann et al., Curr. Opin. Chem. Biol., 17 (6): 982-989 (2013); Mahon et al., Org. Biomol. Chem., 13 (43): 10590-10599 (2015); Yeldell et al., Chem. Soc. Rev., 49 (19): 6848-6865 (2020); Ahmad et al., Nucleic Acids Res., 40 (22): 11777-11783 (2012); and Guo et al., J. Am. Chem. Soc., 137 (34): 11191-11196 (2015)). Rather than using rational design and medchem to optimize multivalency to match the target binding sites, directed evolution methods are being developed to rapidly select optimal multivalent glycan clusters from extremely diverse libraries of 10¹²-10¹³ structures. Previously, carbohydrates were clustered on libraries of DNA aptamer (Tuerk et al., Science, 249 (4968): 505-10 (1990) and Zhou et al., Nat. Rev. Drug Discov., 16 (3): 181-202 (2017)) or peptide backbones, and methods were developed to genetically encode and amplify these libraries (MacPherson et al., Angew. Chem. Int. Ed., 50 (47): 11238-11242 (2011); Temme et al., Chem. Eur. J., 19 (51): 17291-17295 (2013); Temme et al., J. Am. Chem. Soc., 136 (5): 1726-1729 (2014); Horiya et al., In Methods Enzymol., Imperiali, B., Ed. Academic Press, 597:83-141 (2017); Temme et al., Curr. Protoc. Chem. Biol., 7 (2): 73-92 (2015); and Horiya et al., J. Am. Chem. Soc., 136 (14): 5407-5415 (2014)). RNA has a more diverse structural repertoire than DNA, motivating the display of glycan clusters on RNA backbones (Piao et al., RNA, 24 (1): 67-76 (2018); Ostergaard et al, J. Org. Chem., 79 (18): 8877-81 (2014); Batey et al., Angew. Chem. Int. Ed., 38 (16): 2326-2343 (1999); Forconi et al., Chem. Biol., 18 (8): 949-954 (2011); Butcher et al., Acc. Chem. Res., 44 (12): 1302-1311 (2011)). Although RNA is highly susceptible to nuclease degradation, 2′-deoxy-2′-fluoro RNA (F-RNA) exhibits increased serum stability (Cummins et al., Nucleic Acids Res., 23 (11): 2019-2024 (1995); Pieken et al., Science, 253 (5017): 314-7 (1991); Li et al., J. Am. Chem. Soc., 121 (23): 5364-5372 (1999); Duffy et al., BMC Biology, 18 (1): 112 (2020)). However, materials and methods for incorporating F-RNA or other nuclease-resistant modified nucleotides into RNA backbones during the selection process, and subsequent processes for amplification of selected RNA molecules that bind to a target were not available.

The present disclosure is directed to overcoming these and other deficiencies in the art.

### SUMMARY

A first aspect relates to a method for selecting an RNA molecule that binds to a target molecule. The method includes steps of:
providing a pool of oligonucleotide complexes that each comprise a ds-DNA molecule and an RNA molecule, the ds-DNA molecule including a first DNA strand at least partially annealed to a first region of the RNA molecule, whereby a second region of the RNA molecule is free to adopt a secondary structure;
exposing the pool to a target molecule and allowing the second region of the RNA to bind the target molecule; and
selecting from the pool one or more oligonucleotide complexes comprising an RNA molecule having the second region bound to the target molecule;
   wherein the RNA molecule comprises a plurality of modified nucleotides and/or modified ribosyl-phosphate groups within the second region, the first region of the RNA molecule comprises only natural bases and lacks a stable secondary structure, or both.

A second aspect relates to an isolated RNA molecule. The isolated RNA molecule includes a plurality of modified nucleotides, preferably from two to five, wherein the modified nucleotides comprise an oligosaccharide linked to a 2′-fluoro-2′-deoxyuridinyl group (at position 5 of the uridinyl group), wherein the RNA molecule binds to HIV neutralizing antibody 2G12, preferably with an affinity (Kd) of about 40 nM or less.

A third aspect relates to an immunogenic conjugate. The immunogenic conjugate includes an RNA molecule according to the second aspect of disclosure that is covalently or non-covalently bound to an immunogenic carrier molecule.

A fourth aspect relates to a pharmaceutical composition. The pharmaceutical composition includes a pharmaceutically acceptable carrier and an RNA molecule in accordance with the second aspect of the disclosure, or an immunogenic conjugate in accordance with the third aspect of the disclosure.

A fifth aspect includes a method of inducing an immune response in an individual. The method includes the step of administering to an individual an RNA molecule in accordance with the second aspect of the disclosure or an immunogenic conjugate in accordance with the third aspect of the disclosure, wherein said administering is effective to induce an immune response against the oligonucleotide.

A sixth aspect includes a method of inhibiting HIV-1 infection or proliferation. The method includes the step of administering to an individual an RNA molecule in accordance with the second aspect of the disclosure or an immunogenic conjugate in accordance with the third aspect of the disclosure, wherein said administering is effective to induce a neutralizing immune response against HIV-1.

A seventh aspect includes a method for detecting a neutralizing antibody in serum. The method includes the steps of:
providing an RNA molecule in accordance with the second aspect of the disclosure;
contacting the oligonucleotide with serum from an individual; and
detecting whether the oligonucleotide binds specifically to an antibody present in the serum, wherein said detecting is carried out using a label.

Unlike previously reported SELMA (SELection of Modified Aptamers) method (MacPherson et al., Angew. Chem. Int. Ed., 50 (47): 11238-11242 (2011), which is hereby incorporated by reference in its entirety), in which glycan-modified DNA was covalently displayed on unmodified DNA (Zhou et al., Nat. Rev. Drug Discov., 16 (3): 181-202 (2017); Thirunavukarasu et al., J. Am. Chem. Soc., 139 (8): 2892-2895 (2017); Pfeiffer et al., Nat. Protoc., 13 (5): 1153-1180 (2018); Tolle et al., Angew. Chem. Int. Ed., 54 (37): 10971-10974 (2015); and Rohloff et al., Mol. Ther. Nucleic Acids, 3:e201 (2014), which are hereby incorporated by reference in their entirety), the present method (Capture SELMA) utilizes a capture strategy, in which a nascent RNA transcript from a DNA template is non-covalently hybridized to a “capture strand”, and thus travels with its DNA template. The DNA template can be amplified by PCR, regardless of subsequent glycosylation of the RNA, without reverse transcription (Dunn et al., J. Am. Chem. Soc., 42 (17): 7721-7724 (2020); Renders et al., Chem. Comm., 51 (7): 1360-1362 (2015); Gordon et al., ACS Chem. Biol., 14 (12): 2652-2662 (2019), which are hereby incorporated by reference in their entirety).

As described herein, a novel platform is described for design of multivalent carbohydrate cluster ligands by directed evolution, in which serum-stable 2′-fluoro modified RNA (F-RNA) backbones evolve to present the glycan in optimal clusters. This method is validated by the selection of oligomannose (Man₉) glycan clusters from a sequence pool of ˜10¹³ that bind to broadly neutralizing HIV antibody 2G12 with 13 to 36 nM affinities. HIV antibody 2G12 (Trkola et al., J. Virol., 70 (2): 1100-8 (1996), which is hereby incorporated by reference in its entirety) is a target with four glycan binding pockets (Seabright et al., Structure, 28 (8): 897-909.e6 (2020) and Calarese et al., Science, 300 (5628): 2065-2071 (2003), which are hereby incorporated by reference in their entirety) that is of interest in HIV vaccine design (Astronomo et al., Nat. Rev. Drug Discov., 9 (4): 308-24 (2010); Horiya et al., Nat. Chem. Biol., 10 (12): 990-999 (2014); and Bastida et al., Drug Discov. Today Technol., 35-36:45-56 (2020), which are hereby incorporated by reference in their entirety). By demonstrating that this method can evolve tight binders of the HIV antibody 2G12, this supports the belief that the method can be used to generate serum-stable F-RNA aptamers that faithfully mimic native epitopes on HIV gp120. Further, given the success using HIV antibody 2G12, it is fully expected that the method can be adapted for selection of other carbohydrate binding antibodies, as well as carbohydrate binding proteins (CBPs) more generally—which are attractive targets in medicine and biology.

### BRIEF DESCRIPTION OF THE DRAWINGS

FIGS. 1A-1C shows a scheme for library construction. FIG. 1A shows architecture of the Capture SELMA construct. FIG. 1B shows structure of the Man₉ oligomannose glycan.

FIG. 1C shows a capture SELMA cycle. Translucent colors denote F-RNA and solid colors denote DNA. The stem-loop pictured in the RNA region is illustrative of potentially folded RNA, but library sequences are random in the N₂₅ region (red in colored version).

FIGS. 2A-2C show library fraction bound and clone frequency at each round of selection. FIG. 2A shows percent of library bound under selection conditions, quantified by qPCR. FIG. 2B shows control conditions to monitor library fitness: percent bound to constant 150 nM 2G12, quantified by qPCR. FIG. 2C shows clone frequency over final six rounds of selection.

FIGS. 3A-3D show predicted fold of clones 15F1 and 15F3 and mutant sequences with binding data. FIG. 3A shows filter binding curves (left Y-axis) and qPCR binding curves (right Y-axis) for clones 15F1 and 15F3. FIG. 3B shows 15F3 mutant 1 binding (qPCR assay).

FIG. 3C shows 15F1 RNAFold-predicted structure. Shaded/green U=modified uridine, bearing Man₉-cyclohexyl-triazole at uridine 5 position. The asterisk indicates a predicted G-U wobble base pair. X=(A)₁₀ spacer (see Table 2). FIG. 3D shows 15F3 RNAFold-predicted structure.

FIGS. 4A-4B show filter binding of gel-purified 15F3 and stabilized stem variant. FIG. 4A shows filter binding curves for 15F3 crude, heat refolded and gel purified, and 15F3-M1-Mod gel purified. Data were obtained in duplicate. FIG. 4B shows long-stem mutant of 15F3-M1 predicted secondary structure. X=(A)₁₀ spacer, Y=reverse primer (see Table 2).

FIGS. 5A-5B show optimized capture sequence/spacer and Native PAGE analysis of unoptimized and optimized library construction. FIG. 5A shows pre and post-optimization sequences for both the capture sequence (blue in colored) and spacer (orange in colored version).

FIG. 5B shows complex mixture of species was produced during initial attempted construction of the captured F-RNA library (gel, left lane), compared to the optimized preparation (gel, right lane). Once optimized, only the desired productive construct (form E, red box) and an expected byproduct (F-RNA captured on excess rigidified capture strand primer, green box) were present.

FIG. 6 shows a strand swapping test. GlycoRNA/DNA hybrid produced was gel purified, DNase digested, and run on denaturing PAGE (lane 4), alongside controls (lanes 1-3). No 0-click control RNA detected in lane 4, indicating that if strand swapping is occurring, it is below the detection limit of the gel.

FIGS. 7A-B show 15F1 Mutant design/analysis.

FIG. 8 shows a comparison of 15F1 mutant binding towards 300 nM 2G12 using qPCR analysis. None of the 15F1 mutants showed any binding (the highest was Mutant 1 with 3% detected signal compared to the parent clone 15F1 at the tested 300 nM 2G12.

FIGS. 9A-B show 15F3 Mutant design/analysis.

FIG. 10 shows a comparison of 15F3 mutant binding towards 300 nM 2G12 using qPCR analysis. 15F3 mutant 1 bound, but with a lower pM construct bound and a slightly weaker affinity (31±4 nM) as the parent 15F3 clone (19±3 nM) (see FIG. 3B).

FIG. 11 shows a crystal structure of a model GNRA tetraloop. PDB: 1JJ2, The tetraloop is from a 2.4 Angstrom resolution crystal structure obtained of the Haloarcula marismortui Large Ribosomal Subunit (Klein et al., Embo J., 20 (15): 4214-21 (2001), which is hereby incorporated by reference in its entirety). In this particular GNRA tetraloop, the 2′-OH of the second nucleobase points away from the rest of the bases, which would be replaced with a 2′-F in the 15F3. Thus, the 2′-F-pyrimidine of the GNRA sequence (GUGA) could be accommodated in a canonical structure. An important stabilizing hydrogen bond involves the 2′-OH of the first G, which is preserved in the aptamer sequence (GUGA).

FIGS. 12A-12B show the two most prevalent predicted folds of clone 15F3. FIG. 12A shows a major predicted fold, according to RNAFold (Lorenz et al., Algorithms for Molecular Biology, 6 (1): 26 (2011), which is hereby incorporated by reference in its entirety). Predicted ΔG_{folding}=−13.2 kcal/mol. FIG. 12B shows competing predicted fold that completely disrupts the GNRA tetraloop terminated stem. Predicted ΔG_{folding}=−13.4 kcal/mol. The ΔG of each predicted fold was estimated using UNAfold via IDT's website, without accounting for the effect of 2′-fluoro-pyrimidines or glycosylation.

FIG. 13 shows 15F3-M1-Mod design/analysis.

FIG. 14 shows Phosphor imaged filter binding membranes. 2G12 bound aptamer 15F3-M1-Mod is detected on nitrocellulose membrane, where free aptamer is captured on PVDF membrane. 4-fold dilution starting on the left with 1000 nM.

### DETAILED DESCRIPTION

As noted above, the present disclosure relates to a novel method for screening and selecting RNA aptamers, including RNA aptamers that may contain modified RNA analogues.

Nucleic acid aptamers are characterized by a single-strand and have secondary structure that may possess one or more stems (i.e., base-paired regions) as well as one or more non base-paired regions along the length of the stem. These non-base-paired regions can be in the form of a bulge or loop (e.g., internal loop) along the length of the stem(s) and/or a loop at the end of the one or more stem(s) (e.g., hairpin loop). These nucleic acid aptamers possess specificity in binding to a particular target molecule, and they non-covalently bind their target molecule through an interaction such as an ion-ion force, dipole-dipole force, hydrogen bond, van der Waals force, electrostatic interaction, stacking interaction or any combination of these interactions. Aptamers that bind to their target with low nM affinity, or sub-nM affinity, are particularly desirable, and aptamers that possess modified RNA to promote, e.g., stability and resistance to degradation, are also desirable.

Identifying suitable nucleic acid aptamers typically involves selecting aptamers that bind a particular target molecule with sufficiently high affinity and specificity from a pool or library of nucleic acids containing a random region of varying or predetermined length. For example, identifying suitable nucleic acid aptamers of the present disclosure can be carried out using a modification of the established in vitro selection and amplification scheme known as SELEX. The SELEX scheme is described in detail in U.S. Pat. No. 5,270,163 to Gold et al.; Ellington and Szostak, Nature 346:818-822 (1990); and Tuerk and Gold, Science 249:505-510 (1990), each of which is hereby incorporated by reference in their entirety. An established template-primer system (Bartel et al., Cell 67:529-536 (1991), which is hereby incorporated by reference in its entirety) can be adapted to produce RNA molecules having a stretch of about 38-40 random bases sandwiched between 5′ and 3′ constant regions.

Thus, in a first aspect of the present disclosure, a method for selecting an RNA molecule that binds to a target molecule is described herein and illustrated in the accompanying Examples. This method includes providing a pool of oligonucleotide complexes that each comprise a ds-DNA molecule and an RNA molecule, the ds-DNA molecule comprising a first DNA strand at least partially annealed to a first region of the RNA molecule, whereby a second region of the RNA molecule is free to adopt a secondary structure. The method further includes exposing the pool to a target molecule and allowing the second region of the RNA to bind the target molecule. The method further includes selecting from the pool one or more oligonucleotide complexes comprising an RNA molecule having the second region bound to the target molecule; wherein the RNA molecule comprises two to five modified nucleotides or modified ribosyl-phosphate groups within the second region, the first region of the RNA molecule comprises only natural bases and lacks a stable secondary structure, or both.

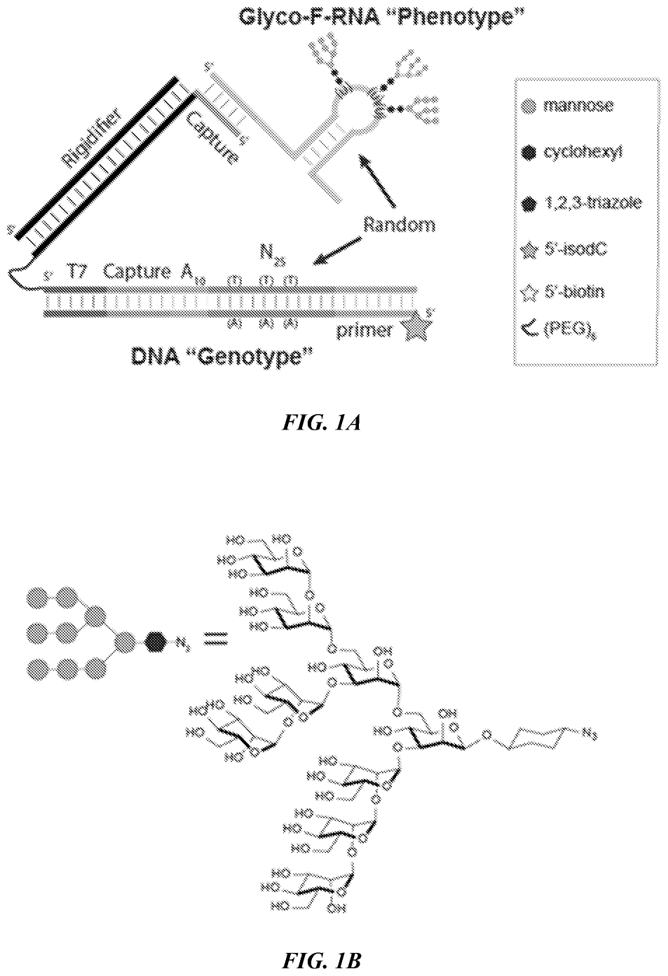

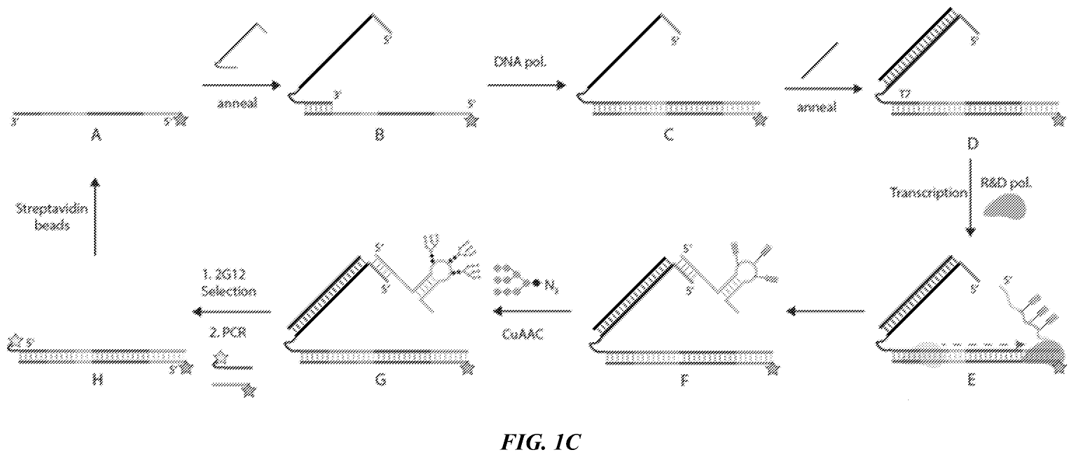

This method for the selection and then regeneration of successive rounds of RNA libraries displayed on their encoded dsDNA is more fully described below and in WO 2018/15470, which is hereby incorporated by reference in its entirety. Briefly, the method involves designing a DNA library such that each DNA duplex can capture its corresponding RNA transcript on a “capture arm” (see WO 2018/15470, which is hereby incorporated by reference in its entirety). By tethering the RNA on a capture arm tethered to its encoding DNA duplex, the RNA can be selected for its binding capability to a target carbohydrate binding polypeptide, thereby selecting the DNA that encodes the RNA for future rounds of maturation and selection. The structure of the dsDNA with its capture arm, complementary rigidifier sequence, and supported F-RNA aptamer bearing one or more glycosylated 2′-deoxy-2′-fluoro-uridinyl bases is shown in FIG. 1A.

Generation of the library starts with singly biotinylated, double stranded DNA (dsDNA) (FIG. 1C, product of Step H), from which the coding strand is removed using streptavidin magnetic beads to afford the single-stranded non-coding strand at FIG. 1C, Step A. The non-coding strand, as shown in FIG. 1A and throughout FIG. 1C includes at its 5′ terminus an unnatural base, e.g., isodC or isodG (star symbol). A capture oligonucleotide is annealed to the non-coding strand (FIG. 1A and FIG. 1C at Steps A, B and extended using Bst 2.0 DNA polymerase (DNAP) to regenerate the dsDNA with a spacer molecule (e.g., 18 atom hexaethyleneglycol (HEG) spacer) that is attached to a single-stranded DNA (ssDNA) extension 5′ of the coding strand (FIG. 1A and FIG. 1C, Step C). In one embodiment, this ss-DNA is a 124-base sequence, although variations of this can be used. The 5′ end of the capture strand contains a sequence that complements the 5′ end of the RNA strand encoded in the DNA library (FIG. 1A). An oligonucleotide is annealed to the capture strand at the region between the HEG spacer and the capture sequence, rigidifying it while maintaining ssDNA at the 5′ end (FIG. 1A and FIG. 1C, Step D). In one embodiment, where the ss-DNA is a 124-base sequence, the rigidifiying oligonucleotide is 82 nts in length. T7 R&DNA polymerase (Sousa et al., EMBO J., 14 (18): 4609-21 (1995), which is hereby incorporated by reference in its entirety) and triphosphates are then added to transcribe F-RNA from the template (FIG. 1C, Step E). In the present invention, 2′-deoxy-2′-fluoro-5-ethynyl-UTP is included to generate the F-RNA that is capable of glycosylation. Presence of the unnatural base at the 5′ end of the non-coding strand causes T7 polymerase to stall (Tanasova et al., ChemBioChem, 16:1212-1218 (2015), which is hereby incorporated by reference in its entirety) at the end of the template (FIG. 1, Step E). Stalling in this manner affords enough time for the 5′ ends of the capture strand and RNA transcript to anneal “intrastructurally” (FIG. 1C, Step F), thereby tethering the RNA strand to its encoding dsDNA. Once the T7 RNAP dissociates and the tethered RNA is allowed to adopt a secondary structure (with its 3′ end free) (FIG. 1C, Step G), the DNA/RNA library is amenable to selection. After selection, amplification of the selected library with a biotinylated forward primer and 5′ isodC reverse primer regenerates the dsDNA library in its original form (FIG. 1C, Step H), completing a single selection cycle. This cycle is intended to be repeated for multiple rounds of DNA/RNA library formation and selection.

It should be appreciated that during amplification of the ds-DNA library from the selected oligonucleotide complexes, a biotinylated forward primer is used and binds to a primer binding site near the 3′ end of the template strand; and a reverse primer containing a terminal non-natural deoxynucleotide base (e.g., isodC or isodG) is used and binds to a primer binding site near the 3′ end of the first portion of the first DNA strand. The amplified ds-DNA library is then recovered.

Based on the foregoing description, it should be apparent that in carrying out the process, the first DNA strand includes a first portion that is (or can be) annealed to a template strand, and a second portion that comprises the 5′ capture region and is tethered at the 3′ end thereof to the 5′ end of the first portion via the linker molecule. The linker molecule is preferably a non-nucleotide linker such as hexaethyleneglycol, polyethylene glycol, an aliphatic hydrocarbon, or a peptide. The linker can also be a nucleotide spacer molecule comprising a plurality of mismatches at the 3′ end of the template strand. It is important for the 5′ capture region to remain unpaired within the ds-DNA molecule (i.e., free of secondary structure). The RNA molecule includes the first region at the 5′ end thereof and the second region extending from the first region to the 3′ end thereof, whereby the first region of the RNA molecule is complementary to and capable of annealing to the 5′ capture region of the first DNA strand. See FIG. 1C, Steps E, F.

In the above description, certain lengths of the oligonucleotides or regions thereof are identified. It should be appreciated by persons of skill in the art that the number of bases in an oligonucleotide or a region thereof, and the base composition thereof, can be adjusted to optimize the behavior of the molecules during the process described above. For instance, the length of the oligonucleotides should be adjusted such that the 5′ ends of the capture strand and RNA transcript are physically close to one another while the RNA transcript remains tethered to the RNAP, thereby promoting the “intrastructural” annealing described above.

When designing the sequences of the capture arm of the DNA display construct, care was taken to minimize secondary structure formation. This was carried out using an iterative approach informed by the use of mFOLD prediction (Zuker, M., Nucleic Acids Res., 31:3406-3415 (2003), which is hereby incorporated by reference in its entirety). In this manner, minimization of secondary structure allowed for annealing of the exemplary 82-base rigidifying oligonucleotide at low temperature (50° C.) despite its high predicted melting temperature (Tₘ˜75° C. at 1 nM concentration). This is a desirable property for optimizing the process, because denaturation of the double stranded DNA library at higher temperatures will destroy the integrity of the library. The capture sequence itself was designed to have minimal secondary structure as well as a high Tₘ with RNA. A key feature for high Tₘ is the lack of A-U base pairs, which are known to destabilize RNA-DNA hybrids considerably (Martin et al., Nucleic Acids Res., 8:2295-2299 (1980), which is hereby incorporated by reference in its entirety). As a result, a capture sequence was designed with Tₘ of ˜70° C. (at 1 nM concentration), a strong non-covalent linkage between the RNA and encoding DNA, capable of forming at the 37° C. transcription temperature. While a Tₘ between the annealed 5′ capture region and first region of the RNA molecule is preferably at least about 60° C., at least about 65° C., or at least about 70° C., sequences having lower Tₘ, such as, at least about 30° C., at least about 40° C., at least about 50° C., or at least about 55° C., can also be used. These lower melting temperatures are less preferred.

Unlike traditional SELEX where the RNA is typically supported on a solid support, i.e., as a partitioning device, the present disclosure involves retaining the RNA tethered to the ds-DNA containing the template. As such, there is no need to use reverse transcription to regenerate the template, because the selected RNA and its template remain tethered throughout each round of selection. In other words, each round of selection for RNA binding to its target necessarily involves selection of the tethered template DNA. This enhances the efficiency of SELEX by avoiding unnecessary steps. It also facilitates the introduction of modified RNA by the T7 polymerase or post-transcription modification. For example, in the SELMA process described above, click-chemistry can be used to introduce desired modification, e.g., glycosylation, onto modified nucleotides prior to selection but typically after FIG. 1C, Step F (MacPherson et al., Angew. Chem., Int. Ed., 50:11238-11242 (2011); Temme et al., Chemistry, 19:17291-17295 (2013); Temme et al., Curr. Protoc. Chem. Biol., 7:73-92 (2015); Temme et al, J. Am. Chem. Soc., 136:1726-1729 (2014), each of which is hereby incorporated by reference in its entirety).

As noted above, in certain embodiments the RNA molecule may have one or more modified nucleotides or modified ribosyl-phosphate groups. These modified nucleotides may be selected from the group of 2′-fluoro-ribonucleotides, 2′-amino-ribonucleotides, 2′-O-methyl-ribonucleotides, 5′-iodo-ribonucleotides, 5′-bromo-ribonucleotides, and alkyne-modified ribonucleotides, as well as deoxyribonucleotide variants thereof. Furthermore, the modified ribosyl-phosphate group may include a phosphorothioate-linked nucleotide.

As is well known in the art, different conditions can be used during amplification and selection to encourage the generation of high affinity aptamers. For example, optimization of aptamers can be achieved during (re) selection by using rigorous washing conditions in all steps, including the use of high temperature (37° C. or 45° C.) washing buffers, mild denaturants, and low salt and high salt washes, etc. The proposed stringent washing conditions are intended to select for aptamers that bind more tightly to the target molecules, and thereby improve the overall affinity. An additional benefit of generating RNA aptamers that bind with higher affinity to the target is that lower concentrations of therapeutic agents of the present disclosure will be needed for therapeutic in vivo applications.

Another method to use for aptamer optimization is the use of a smaller bias during doping. For example, the library can be doped with a 2:1:1:1 ratio instead of 5:1:1:1. This will result in more library members being substantially different from the parent aptamer, allowing for more rapid evolution and selection of the highest affinity binders.

Modified versions of T7 polymerase can also be utilized. For example, a variety of selected T7 mutant polymerases have been identified which allow for efficient incorporation of 2′-O-methyl nucleotides and 2′-fluoro modified pyrimidines (see Meyer et al., Nucl. Acids Res. 43 (15): 7480-748 (2015), which is hereby incorporated by reference in its entirety).

In the procedure identified in FIG. 1C, the rigidifying oligonucleotide serves to increase the likelihood that the RNA and capture strand will come into contact and anneal. Within the capture strand, the HEG spacer is used to maintain the ssDNA nature of the 5′ capture sequence. Without this spacer, the 3′ end of non-coding strand would be extended in steps B→C, creating a full-length complement of the capture arm that would block its ability to anneal to RNA. Any other suitable spacer molecule can be used as long as it is sufficient to block extension of the non-coding strand at its 3′ end. In one embodiment, the template strand includes a random nucleotide sequence region in between first and second fixed nucleotide sequence regions respectively located 3′ and 5′ of the random nucleotide sequence, and the first fixed nucleotide sequence region comprises a poly-A sequence. In another embodiment, the modified nucleotides include an oligosaccharide linked to a 2′-fluoro-2′-deoxyuridinyl group (at position 5 of the uridinyl group).

A further aspect relates to an isolated RNA molecule. The isolated RNA molecule includes from two to five modified nucleotides, wherein the modified nucleotides comprise an oligosaccharide linked to a 2′-fluoro-2′-deoxyuridinyl group (at position 5 of the uridinyl group), wherein the RNA molecule binds to HIV neutralizing antibody 2G12, preferably with an affinity (Kd) of about 40 nM or less.

In another embodiment, the isolated RNA molecule includes from two to five modified nucleotides, wherein the modified nucleotides comprise an oligosaccharide linked to a 2′-fluoro-2′-deoxyuridinyl group (at position 5 of the uridinyl group), wherein the RNA molecule binds to a carbohydrate-binding and neutralizing antibody, preferably with an affinity (Kd) of about 100 nM or less.

The RNA of the isolated RNA molecule may include any suitable number of modified nucleotides. For example, there may be 1 modified nucleotide, 2 modified nucleotides, 3 modified nucleotides, 4 modified nucleotides, 5 modified nucleotides, or more than 5 modified nucleotides. In one embodiment, the RNA of the isolated RNA molecule includes two to four modified nucleotides.

In one embodiment, the oligosaccharide is a Man₉ group linked via a cyclohexyl-triazole linker to the uridinyl 5 position. The RNA molecule may bind to the antibody (e.g., antibody 2G12) with any suitable Fbₘₐₓ, for example, the Fbₘₐₓ may be about 7%, about 8%, about 9%, about 10%, about 15%, about 20%, about 25%, about 30%, about 35%, about 40%, about 45%, about 50%, about 55%, about 60%, about 65%, about 70%, about 75%, or greater than 75%. In one embodiment, the RNA molecule binds to antibody 2G12 with an Fbₘₐₓ of 7% or greater. In one embodiment, the RNA molecule binds to antibody 2G12 with an Fbmax of 20% of greater, 30% or greater, or 40% or greater. In one embodiment, the RNA molecule binds to antibody 2G12 with an Fbmax of 50% of greater. Similarly, the RNA molecule may bind to the antibody (e.g., antibody 2G12) with any suitable Kd, for example the Kd may be about 20 nM, about 19 nM, about 18 nM, about 17 nM, about 16 nM, about 15 nM, about 14 nM, about 13 nM, about 12 nM, about 10 nM, about 9 nM, about 8 nM, about 7 nM, about 6 nM, about 5 nM, about 4 nM, about 3 nM, about 2 nM, about 1 nM, or less than about 1 nM. In one embodiment, the RNA molecule binds to antibody 2G12 with a Kd of 20 nM or less.

The RNA molecule described herein may, in one embodiment, include a stem-loop structure comprising two of the modified nucleotides at adjacent positions in the stem and one of the modified nucleotides in the loop. In one embodiment, the RNA molecule comprises the sequence of 5′-X-GCGCCCAGGNGANNGGGCGC-Y-3′ (SEQ ID NO: 1) where X is optionally a poly-A sequence and Y is optionally-AAGCGACACAAAGCCCGG-3′ (SEQ ID NO: 2), which has the predicted structure of

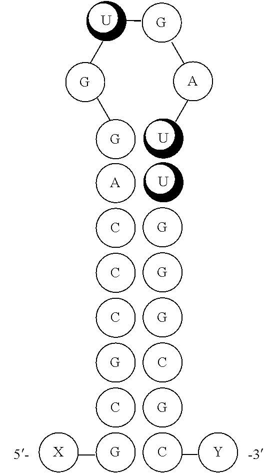

A further aspect relates to an immunogenic conjugate. The immunogenic conjugate includes an RNA molecule in accordance with those described herein that is covalently or non-covalently bound to an immunogenic carrier molecule.

The immunogenic carrier molecule may be selected from one or more of bovine serum albumin, chicken egg ovalbumin, keyhole limpet hemocyanin, tetanus toxoid, diphtheria toxoid, thyroglobulin, a pneumococcal capsular polysaccharide, CRM 197, and a meningococcal outer membrane protein.

A further aspect of the present disclosure relates to one or more kits useful for carrying out the methods of the present disclosure. Each of the previously described components can be packaged into a kit, including one or more of the following: a ds-DNA optionally having a non-natural nucleic acid molecule at a 5′ end of a template strand; a DNA capture strand that anneals to the template strand and has a first portion primer sequence tethered via a linker molecule to a second portion that includes a 5′ capture region; a DNA rigidifier strand that is capable of annealing to the second portion of the DNA capture strand; and optionally one or more of a DNA polymerase, dNTPs, an RNA polymerase, rNTPs or modified rNTPs of the type described above, one or more buffer solutions, and instructions for carrying out the process described herein. Quantities of the above reagents suitable for performing up to 5, 6, 7, 8, 9, or 10 or more rounds of selection and amplification can be provided.

Once high affinity RNA aptamers are identified, the secondary structure of each primary RNA aptamer can be predicted by computer programs such as MulFold or mFOLD (Jaeger et al., Proc. Natl. Acad. Sci. USA, 86:7706-7710 (1989); Zuker, Science, 244:48-52 (1989), each of which is hereby incorporated by reference in its entirety). Mutational studies can be conducted by preparing substitutions or deletions to map both binding sites on the RNA aptamer and its target molecule, as well as to further enhance aptamer binding affinity, as described in the accompanying Examples.

Aptamers generated from SELEX experiments can be optimized to produce second generation aptamers with improved properties (Eaton et al., Bioorg. Med. Chem. 5:1087-1096 (1997), which is hereby incorporated by reference in its entirety). Through successive rounds of affinity maturation of a primary SELEX clone, it is possible to obtain aptamers that possess improved affinity for their target as compared to the original clone. Therefore, prior to using aptamers in cell-based experiments, each aptamer can be optimized using the following considerations:
Find the minimal aptamer sequence within the SELEX clone to identify the domain to subject to affinity maturation. This will lead to more desirable, smaller aptamers, which should be better for fusing with alternative functional domains and for in vivo delivery;
It is important to know if the aptamers are selective for their intended target molecule or if they bind indiscriminately to structurally related, non-target molecules; and
Additionally, forming targeted delivery vehicles includes fusing the aptamer sequence with one or more nucleic acid or non-nucleic acid based delivery molecules, optionally with one or more additional aptamers for the same target molecule. This will enhance internal delivery of the aptamers to cell targets of interest and decrease their off-rate from the target molecule of interest. The formation of these fusion molecules should be possible without impacting the aptamer ability to bind specifically to a particular target molecule of interest.

As used herein, “nucleic acid” includes both DNA and RNA, in both D and L enantiomeric forms, as well as derivatives thereof (including, but not limited to, 2′-fluoro-, 2′-amino, 2′O-methyl, 5′iodo-, and 5′-bromo-modified polynucleotides). Nucleic acids containing modified nucleotides (Kubik et al., J. Immunol. 159:259-267 (1997); Pagratis et al., Nat. Biotechnol. 15:68-73 (1997), each which is hereby incorporated by reference in its entirety) and the L-nucleic acids (sometimes termed Spiegelmers®), enantiomeric to natural D-nucleic acids (Klussmann et al., Nat. Biotechnol. 14:1112-1115 (1996) and Williams et al., Proc. Natl. Acad. Sci. USA 94:11285-11290 (1997), each which is hereby incorporated by reference in its entirety), and non-natural bases are used to enhance biostability. In addition, the sugar-phosphate backbone can be replaced with a peptide backbone, forming a peptide nucleic acid (PNA), other natural or non-natural sugars can be used (e.g., 2′-deoxyribose sugars), or phosphothioate or phosphodithionate can be used instead of phosphodiester bonds.

Increasing the number of aptamer domains in a single molecule, i.e., a multivalent aptamer, also is shown to decrease the dissociation of the aptamer from its target. Therefore, the present disclosure also contemplates aptamer constructs that include a series of target molecule-binding aptamers that are joined together by linking nucleotides sequences that do not adversely affect the secondary structure of the individual aptamer domains. Multivalent aptamers of this type can be constructed as described in Shi et al., Proc Natl Acad Sci USA 96 (18): 10033-10038 (1999) (describing pentavalent aptamer constructs); Xu and Shi, Nucl Acids Res 37 (9): 1-9 (2009) (describing di-dimeric aptamer construct with three-way junction); U.S. Patent Application Publ. No. 20050282190 to Shi et al. (describing multimeric aptamer constructs containing three-way junctions), each of which is hereby incorporated by reference in its entirety. Before joining two functional RNA molecules, it is often beneficial to first predict the secondary structures of the chimeric nucleic acid molecule to ensure that their combination is unlikely to disrupt their secondary structures. Secondary structure predictions can be performed using a variety of software including, without limitation, RNA Structure Program (Dr. David Mathews, University of Rochester) and MFold (Dr. Michael Zuker, The RNA Institute, SUNY at Albany), among others. In certain embodiments, the aptamer domains can bind to the same binding site on the target molecule. In alternative embodiments, the aptamer domains can bind to more than one distinct site on the target molecule.

Sequences that stabilize the aptamer can also be introduced. One example of a stabilization sequence is an exonuclease-blocking sequence linked to an aptamer sequence. In particular, a stable tetra-loop near the 3′ end of the aptamer can be engineered. Because of its highly stacked and relatively inaccessible structure, the UUCG tetra-loop (Cheong et al., Nature 346:680-682 (1990), which is hereby incorporated by reference in its entirety) can be used to stabilize nucleic acid molecules against degradation by 3′ exonucleases and to serve as a nucleation site for folding (Varani, Annu. Rev. Biophys. Biomol. Struct. 24:379-404 (1995), which is hereby incorporated by reference in its entirety). Structurally, this type of loop is also used as a “U-turn” to close a stem region to make the strand continuous as a single molecular entity. Suitable U-turns for RNA include, without limitation, members of the UNCG and GNRA tetraloop families (Varani, Annu. Rev. Biophys. Biomol. Struct. 24:379-404 (1995), which is hereby incorporated by reference in its entirety). Suitable U-turns for DNA include, without limitation, members of the GNRA tetraloop family (Varani, “Annu. Rev. Biophys. Biomol. Struct. 24:379-404 (1995), which is hereby incorporated by reference in its entirety).

Another example of a stabilization sequence is an “S35 motif” which yields a virtually closed structure resistant to nucleolytic degradation. The S35 motif, constructed by creating complementary 5′ and 3′ ends, has been shown to cause an over 100-fold increase in accumulation of a tRNA-ribozyme chimerical transcript in stably transduced cell lines (Thompson et al., Nucleic Acids Res. 23:2259-2268 (1995), which is hereby incorporated by reference in its entirety). Its use with in vivo aptamer expression has been demonstrated previously. See Shi et al., Mol Cell Biol 17 (5): 2649-2657 (1997); U.S. Pat. No. 6,458,559 to Shi et al., each of which is hereby incorporated by reference in its entirety.

Having prepared and optimized the aptamer obtained in accordance with the present disclosure, the aptamers can be formulated as one component of a molecular delivery agent that also includes a cell targeting component that is covalently or non-covalently linked to the nucleic acid aptamer molecule. The cell targeting component can be another aptamer, an antibody or binding fragment thereof, or an antibody mimic that is specific for, e.g., a cell surface molecule that is present on the target cell type. According to this approach, the cell targeting component binds to a cell surface molecule on the target cell type, and the molecular delivery agent is taken up by the cell and the nucleic acid aptamer molecule is internalized into the cell where it can bind to and interact with the target molecule of interest to modify (typically inhibiting) its activity.

A further aspect relates to a pharmaceutical composition. The pharmaceutical composition comprises a pharmaceutically acceptable carrier and an RNA molecule in accordance with those described herein, or an immunogenic conjugate comprising the RNA molecule covalently or non-covalently bound to an immunogenic carrier molecule.

Pharmaceutical compositions suitable for injectable or parental use (e.g., intravenous, intra-arterial, intramuscular, etc.) or intranasal use may include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. Suitable carriers and/or excipients, include, but are not limited to sterile liquids, such as water, saline solutions, and oils, with or without the addition of a surfactant and other pharmaceutically and physiologically acceptable carriers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

The pharmaceutical compositions can also include one or more additives or preservatives, or both. The pharmaceutical composition may, in one embodiment, further include any suitable adjuvant.

Effective amounts of the aptamer or molecular delivery agent may vary depending upon many different factors, including mode of administration, target site, physiological state of the patient, other medications administered, and whether treatment is prophylactic or therapeutic. Treatment dosages need to be titrated to optimize safety and efficacy. The amount of aptamer or molecular delivery agent depends on the frequency of administration, the rate of clearance, and the patient population (e.g., adult or child). For example, dosages may vary from 1 μg-5 mg per dose and more usually from 5-1000 μg per dose for human administration.

A further aspect includes a method of inducing an immune response in an individual. The method includes administering to an individual an RNA molecule in accordance with those described herein or an immunogenic conjugate comprising the RNA molecule covalently or non-covalently bound to an immunogenic carrier molecule, wherein said administering is effective to induce an immune response against the oligonucleotide.

Administering of the present aspect is, in one embodiment, effective to induce a carbohydrate-binding, neutralizing antibody response. Moreover, the induced carbohydrate-binding, neutralizing antibody response is, one embodiment, protective against HIV-1.

Administering of this aspect may be carried out in accordance with the previously described aspect. In one embodiment, administering is carried out orally, parenterally, subcutaneously, intravenously, intramuscularly, intraperitoneally, by intranasal instillation, by implantation, by intracavitary or intravesical instillation, intraarterially, intralesionally, transdermally, intra- or peri-tumorally, by application to mucous membranes, or by inhalation. The administering may be repeated and suitable number of times.

The dosage of the oligonucleotide or immunogenic conjugate is administered at an amount suitable for the effective result. For example, the dose may be between 0.1 μg and 1,000 mg. The dose may be, for example, about 0.1 μg, about 0.5 μg, about 1 μg, about 10 μg, about 50 μg, about 75 μg, about 100 μg, about 500 μg, about 600 μg, about 700 μg, about 800 μg, about 900 μg, about 1 mg, about 2 mg, about 3 mg, about 4 mg, about 5 mg, about 10 mg, about 25 mg, about 50 mg, about 75 mg, about 100 mg, above 100 mg, or any amount therebetween. In one embodiment, the dose is between about 1 μg to about 5 mg.

As used herein, the terms “individual”, “subject”, or “patient” are used interchangeably, and means any animal, including mammals such as mice, rats, other rodents, rabbits, dogs, cats, swine, sheep, cattle, horses, or primates, such as humans. In one embodiment, the individual is a human.

As used herein, the phrase “in need thereof” means that the subject has been identified as having a need for the particular method described herein. In some embodiments, the identification can be by any means of diagnosis. In any of the methods described herein, the subject can be in need thereof.

A further aspect includes a method of inhibiting HIV-1 infection or proliferation. The method includes administering to an individual an RNA molecule in accordance with those described herein or an immunogenic conjugate comprising the RNA molecule covalently or non-covalently bound to an immunogenic carrier molecule, wherein said administering is effective to induce a neutralizing immune response against HIV-1.

A further aspect includes a method for detecting a neutralizing antibody in serum. The method includes providing an RNA molecule in accordance with those described herein; contacting the oligonucleotide with serum from an individual; and detecting whether the oligonucleotide binds specifically to an antibody present in the serum, wherein said detecting is carried out using a label

### EXAMPLES

The following Examples are presented to illustrate various aspects of the disclosure, but are not intended to limit the scope of the claimed invention.

### Materials and Methods for Examples

All synthetic DNA oligonucleotides were purchased from Integrated Technologies (Coralville, IA), and purified via denaturing polyacrylamide gel electrophoresis (PAGE). A complete list of oligos and primers for capture-SELMA is in Table 1. BST 2.0 warm start DNA polymerase, T4 polynucleotide kinase, dNTPs, rNTPs, Exonuclease I, and BSA was purchased from New England Biolabs. Hydrophilic streptavidin M270-dynabeads, Protein-A and G dynabeads, and a TOPO-TA cloning kit were purchased from Invitrogen. All qPCR analyses were performed on a MIC (magnetic induction cycler) qPCR from Bio Molecular Systems using qPCRBio SyGreen Blue Mix Lo-Rox (cat #SMP17-505). [γ-³²P]-ATP was purchased from PerkinElmer. Centri-spin 20 microcentrifuge size exclusion columns were purchased from Princeton Separations. Sybr Gold nucleic acid gel stain was purchased from Life Technologies.

TABLE 1
Commercially purchased oligonucleotides

| | | SEQ ID |
|---|---|---|
| Name | Sequence | NO: |
| Library^{†} | 5′CCGGGCTTTGTGTCGCTINNNNNNNNNNNNNNNNNNNNNNNNNTTTT | 3 |
| | TCTCTCTTCCCTCCTTCTCTCCTATAGTGAGTCGTATTACAGTTG | |
| CapArm* | 5′CTCTCTTCCCTCCTTCTCTCCTTTTTCAACACCACAGACCAGTATAC | 4, 5 |
| | CCAGAAATGACGCAAGCATAGACAAACGATTTAGACATGAGTGCCCGAC | |
| | ACAACGAACAAGCTTTTTTTTTA-hexaethyleneglycol | |
| | SPACER-CAACTGTAATACGACTCACTATAGGAGA | |
| Rigidifier | 5′GCTTGTTCGTTGTGTCGGGCACTCATGTCTAAATCGTTTGTCTATGC | 6 |
| | TTGCGTCATTTCTGGGTATACTGGTCTGTGGTGTTG | |
| Biotin For | 5′Biotin-CAACTGTAATACGACTCACTATAGGAGA | 7 |
| primer | | |
| Phosphate | 5′Phosphate-CAACTGTAATACGACTCACTATAGGAGA | 7 |
| For primer | | |
| Forward | 5′CAACTGTAATACGACTCACTATAGGAGA | 7 |
| primer | | |
| IsodC Rev | 5′isodC-CCGGGCTTTGTGTCGCTT | 8 |
| primer | | |
| 3-U | 5′CCGGGCTTTGTGTCGCTTTATCCGTAGGTTGCACCGTGGGTCTTTTT | 9 |
| | TTTTTCTCTCTTCCCTCCTTCTCTCCTATAGTGAGTCGTATTACAGTTG | |
| 0-U | 5′CCGGGCTTTGTGTCGCTTTGTCCGTGGGTTGCGCCGCGGCCGCTTTT | 10 |
| | TTTTTCTCTCTTCCCTCCTTCTCTCCTATAGTGAGTCGTATTACAGTTG | |
| 15F1-M1 | 5′CCGGGCTTTGTGTCGCTTTCCACAGGGGGCCCCTGTTCCGTCGTTTT | 11 |
| | TTTCTCTCTTCCCTCCTTCTCTCCTATAGTGAGTCGTATTACAGTTG | |
| 15F1-M2 | 5′CCGGGCTTTGTGTCGCTTTCCACTGGGGGCCCCTGTTCCGTCGATTT | 12 |
| | TTTCTCTCTTCCCTCCTTCTCTCCTATAGTGAGTCGTATTACAGTTG | |
| 15F1-M3 | 5′CCGGGCTTTGTGTCGCTTTCCTCAGGGGGCCCCTGTTCCGTCGATTT | 13 |
| | TTTCTCTCTTCCCTCCTTCTCTCCTATAGTGAGTCGTATTACAGTTG | |
| 15F3-M1 | 5′CCGGGCTTTGTGTCGCTTGCCCCAATCACCTGGGTTCCCTCTGTTTT | 14 |
| | TTTTCTCTCTTCCCTCCTTCTCTCCTATAGTGAGTCGTATTACAGTTG | |
| 15F3-M2 | 5′CCGGGCTTTGTGTCGCTTGCCCCAATCTCCTGGGTTCCCTATGTTTT | 15 |
| | TTTTCTCTCTTCCCTCCTTCTCTCCTATAGTGAGTCGTATTACAGTTG | |
| 15F3-M3 | 5′CCGGGCTTTGTGTCGCTTGCCCCAGTCACCTGGGTTCCCTATGTTTT | 16 |
| | TTTTCTCTCTTCCCTCCTTCTCTCCTATAGTGAGTCGTATTACAGTTG | |
| 15F3-M4 | 5′CCGGGCTTTGTGTCGCTTGCCGCCATCACCGGCGTTCCCTATGTTTT | 17 |
| | TTTTCTCTCTTCCCTCCTTCTCTCCTATAGTGAGTCGTATTACAGTTG | |
| 15F3-Mut- | 5′CCGGGCTTTGTGTCGCTTGCGCCCAATCACCTGGGCGCTTTTTTTTT | 18 |
| 1-Mod | TCTCTCTTCCCTCCTTCTCTCCTATAGTGAGTCGTATTACAGTTG | |

^{†}In the Library, the N₂₅ Randomized region is (N1:15%/28%/29%/28%:A/G/C/T).
*In the CapArm, the capture sequence is shown in bold, and the rigidifier binding site is underlined.

Nitrocellulose membranes (0.45 μm) were purchased from Biorad. PVDF membranes (0.45 μm, immobilon-FL) were purchased from Millipore. Water was purified with a Milli-Q Ultrapure water purification system. Prepared buffers were sterilized by filtration through 0.22 μm syringe filters obtained from Millipore. All other reagents were purchased from National Diagnostics, Sigma-Aldrich, Acros Organics, New England Biolabs, or Fisher and used without further purification unless otherwise noted.

SELMA Library construction—A starting random double stranded DNA (dsDNA) library was produced by standard PCR of a synthetic random single stranded (ssDNA) library using a biotinylated forward primer and isodC reverse primer with Phusion hot start II DNA polymerase (Thermo: F549L) (Table 1 shows sequence information). A 400 μL PCR reaction consisted of 160 pmol biotinylated forward primer and isodC reverse primer, 20 pmol negative strand template, 1× high fidelity (HF) buffer, 8 units Phusion hot start II DNA polymerase, 15 μg/mL BSA, 200 μM each dNTP and cycling at 98° C. for 30 seconds followed by 6 cycles of 98° C. for 10 seconds, 65° C. for 10 seconds, and 72° C. for 10 seconds. Following PCR amplification, the reaction was quenched by adding 5 μL of 0.5M EDTA and 20 μL of 5M NaCl. To remove the biotinylated strand, the crude PCR product was incubated with 2 mg (200 μL) hydrophilic streptavidin magnetic beads (SMB) (Thermo: 65305) for 30 minutes. The beads were washed thrice with SMB wash buffer (10 mM Tris pH 7.5, 500 μM EDTA, 1M NaCl, 0.2% Tween-20) and then resuspended in 100 μL 0.15M NaOH for 4 minutes followed by application of a magnetic separator and transfer of the supernatant to a tube containing 11 μL 100 mM Tris pH 8 and 13 μL 1.25M acetic acid.

For installation of the capture arm into the random library, 6 pmol ss-isodC library (above) was combined with 12 pmol capture arm primer, diluted to 96 μL in 1× thermopol buffer. The mix was heated to 95° C. for 1 minute and cooled to 57° C. at a rate of 1° C./second, followed by incubation at 57° C. for 1 minute. The tube was placed on ice and 1 μL 10 mM dNTPs were added followed by the addition of 8 units BST 2.0 warmstart DNA polymerase. The reaction was incubated at 60° C. for 4 minutes, followed by cooling on ice for 1 minute before being buffer exchanged into BE buffer (10 mM Tris pH 7.5, 2 mM MgSO₄) using a size exclusion Centri-spin 20 column (Princeton Separations: CS-201).

To rigidify the capture arm with a complementary 83mer oligonucleotide, 60 μL of the buffer-exchanged product was combined with 20 μL R&DNA T7 polymerase 5× reaction buffer and 7.6 pmol rigidifier oligonucleotide, diluted to 85 μL before being heated to 50° C. for 5 minutes. To this solution containing rigidified capture arm was then added 10 μL 100 mM DTT, 2 μL 10 mM modified NTP mix (5-ethynyl-2′-fluoro-dUTP, 2′-fluoro-dCTP, rATP, rGTP) followed by 150 units of R&DNA T7 polymerase; the tube was then heated to 37° C. for 12 minutes. Transcription samples were then immediately buffer exchanged on centri-spin 20 spin columns hydrated with CBE buffer (10 mM HEPES pH 8, 2 mM MgSO₄).

CuAAC—Glycosylation of the library using CuAAC was performed similar to previously reported (Temme et al., JACS 136 (5): 1726-1729 (2014), which is hereby incorporated by reference) with slight modifications. All stock reagents were prepared in water and stored-20° C. 202 nmol sodium ascorbate solution was placed in a capless 0.5 mL microfuge tube (tube 1). 90 nmol CuSO₄ and 108 nmol THPTA solutions were added to a second capless tube (tube 2). To a third tube (tube 3), 90 μL (3.3 pmol) transcribed construct (above) and 50 nmol Man₉-cyclohexyl-N₃ solution. All 3 tubes were concentrated via speed-vac until tubes 1 and 2 were dry and tube 3 was roughly 20 μL. All tubes were then transferred to a 2-neck flask containing positive N₂ flow. 5 μL of degassed water was used to redissolve the residue in tube 2 (Cu/THPTA) and this solution was transferred to tube 1 to redissolve the sodium ascorbate and reduce the Cu(II) to Cu(I) before being transferred to tube 3 containing the library and azide. The reaction was run under slight positive N₂ pressure for 3 hours, then diluted to 50 μL and buffer exchanged into BE buffer.

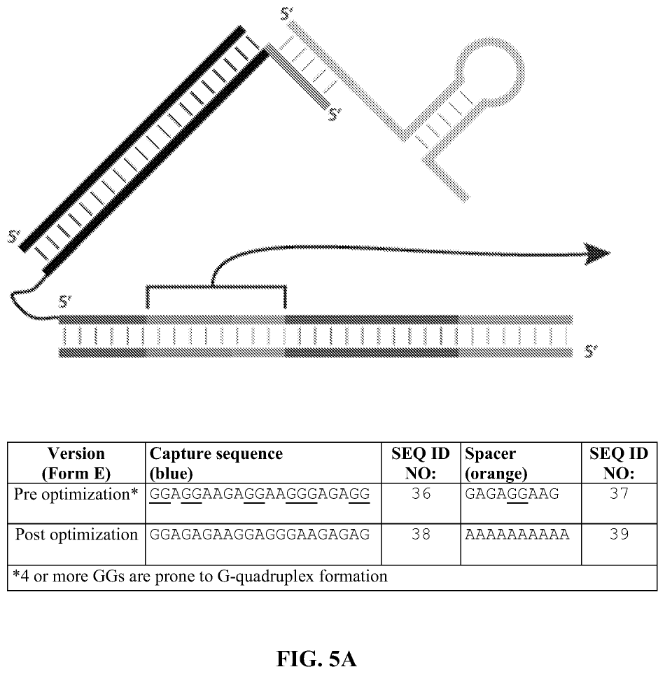

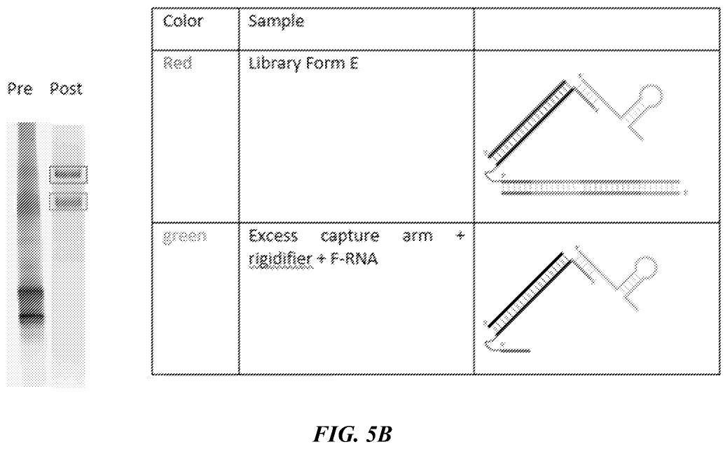

Strand Swapping Test—To determine the possible extent of RNA strand swapping between captured RNA and free RNA transcripts in solution, transcription/capture and CuAAC glycosylation of a sequence containing 3 glycosylation sites was performed in the presence of an RNA sequence containing no glycosylation sites, transcribed from a different template sequence that lacks a capture arm. The templates were assembled separately and mixed 1:1 before transcription. Transcription was performed, followed by CuAAC glycosylation and native-PAGE purification of the fully built construct band (as in FIG. 5B, band in box). When purified construct was DNase-digested and analyzed on denaturing urea-PAGE no unglycosylated RNA was detected in the gel.

TABLE 2
Primer Sequences and Glycosylation Sites

| | | | | # |
|---|---|---|---|---|
| | | | | glyco- |
| Sequence | Sequence | | | sylation |
| name | color | Primer | Sequence | sites |
| 3-U | Red | Capture | AGACCCACGGUGCAA | 3 |
| | | arm | CCUACGGAUA | |
| | | | (SEQ ID NO: 19) | |
| 0-U | Yellow | Forward | GCGGCCGCGGCGCAA | 0 |
| | | primer | CCCACGGACA | |
| | | | (SEQ ID NO: 20) | |

Selection-All 2G12 selections were performed similar to previously reported (Temme et al., Journal of the American Chemical Society 136 (5): 1726-1729 (2014), which is hereby incorporated by reference), with the following modifications. Selection at rounds 1-8 were performed at room temperature followed with the remaining rounds being performed at 37° C. In all rounds except round 1, the library was first incubated with prewashed Protein A/G magnetic 0.75 mg Dynabeads for 30 min, and then transferred to a second tube. The selection then proceeded by first mixing library with 2G12 in solution at desired target concentrations for 30 minutes, followed by the addition of 1.5 mg prewashed Protein A or G magnetic Dynabeads for 30 minutes at each round's selection temperature. Beads were then separated from supernatant and washed (see Table 3 for wash details per round) with bind buffer+Tween (20 mM Tris pH 7.5, 150 mM NaCl, 2 mM MgSO₄, 0.02% tween-20) prewarmed to each specific round's selection temperature. For elution of bound library members, beads were resuspended in 3.5M MgCl₂ (50 μL) and rotated for 5 minutes, before being separated from the magnetic beads and buffer-exchanged 2× into BE using Centri-spin 20 microcentrifuge columns (Princeton Separations). To monitor each round's recovery and obtain the optimal PCR cycle number for regeneration of library, qPCR was performed on buffer-exchanged recovered construct. Briefly, 1 μL of eluted construct was analyzed in iTaq Universal SYBR Green Supermix (Biorad: 1725121) with 1 μM 2.0RR-Bio-F-P and 2.0RR-IsodC-R-P primers, according to manufacturer's directions. qPCR conditions were as follows: 95° C. for 2 minutes for an initial denaturation/activation of the hot start polymerase, followed by a two-step PCR method cycling between 95° C. for 5 seconds and 60° C. for 20 seconds for 40 cycles. The % recovery of each round was estimated by analyzing a serial dilution series of known concentrations of template simultaneously with the round recovered samples. Eluted library was amplified via PCR with Phusion hot start II DNA polymerase, utilizing similar conditions as previously mentioned (Library Construction section) choosing cycle number based off qPCR results. To remove the biotinylated strand, the crude PCR product was incubated with 1 mg (100 μL) hydrophilic streptavidin magnetic beads (SMB) (Thermo: 65305) for 30 minutes. The beads were washed thrice with SMB wash buffer (10 mM Tris pH 7.5, 500 μM EDTA, 1M NaCl, 0.2% Tween-20) and then resuspended in 50 μL 0.15M NaOH for 4 minutes followed by application of a magnetic separator and transfer of the supernatant to 5.5 μL 100 mM Tris pH 8 and 6.5 μL 1.25M acetic acid.

TABLE 3
Selection conditions and qPCR-estimated % bound per round

| Selection | 2G12 | | % bound | % bound | Antibody | Wash | Number |
|---|---|---|---|---|---|---|---|
| Round | conc. (nM) | Temp | (Selection) | (mini selection) | capture beads | volume (μL) | of washes |
| 1 | 150 | RT | 0.67 | — | Protein A | 200 | 3 |
| 2 | 150 | RT | 0.035 | — | Protein A | 200 | 3 |
| 3 | 150 | RT | 0.15 | — | Protein A | 200 | 3 |
| 4 | 150 | RT | 0.13 | — | Protein A | 200 | 4 |
| 5 | 150 | RT | 0.16 | — | Protein A | 200 | 4 |
| 6 | 75 | RT | 0.85 | 1.3 | Protein A | 200 | 4 |
| 7 | 25 | RT | 0.74 | 2.6 | Protein A | 300 | 4 |
| 8 | 25 | RT | 1.74 | 5.9 | Protein A | 300 | 4 |
| 9 | 25 | 37 | 0.14 | 12.1 | Protein A | 300 | 4 |
| 10 | 25 | 37 | 0.48 | 10.9 | Protein A | 300 | 4 |
| 11 | 25 | 37 | 0.97 | 8.8 | Protein A | 300 | 4 |
| 12 | 25 | 37 | 1.56 | 8.5 | Protein A | 300 | 4 |
| 13 | 25 | 37 | 0.96 | 12.3 | Protein G | 300 | 4 |
| 14 | 10 | 37 | 0.44 | 11.4 | Protein G | 300 | 4 |
| 15 | 10 | 37 | 0.47 | 9.8 | Protein G | 400 | 4 |
| 16 | 5 | 37 | 0.80 | 9.3 | Protein G | 400 | 4 |
| 17 | 2 | 37 | 0.23 | 5.8 | Protein G | 400 | 4 |
| 18 | 2 | 37 | 0.36 | 14.7 | Protein G | 400 | 4 |

General qPCR protocol—General qPCR cycle information unless otherwise described are as follows: 2 min 95° C. initial denaturation followed by 40 cycles of two step PCR cycling between 95° C. (5 sec.) and 60° C. (20 sec.). Melting analysis was also performed after each amplification by incubating at 65° C. (1 min.) followed by increase to 95° C. at 0.3° C./sec.

qPCR analysis of “mini-selection” and actual selection—Every round of selection was monitored with qPCR to obtain information regarding selection progress. The optimal cycle number to recover the bound construct was obtained, and % bound was calculated for both a “mini-selection” (a small portion of the library subjected to the Round 1 selection conditions) as well as the main selection (most of the library subjected to different conditions depending on each round). Following the qPCR analysis, a melting point analysis of amplified library was used to qualitatively assess the diversity of the library.

Melting analysis via qPCR to estimate convergence of library-After 40 cycles of amplification, each samples' melting point was measured scanning from 65° C. to 95° C. at a rate of 0.3° C./second using the qPCR instrument. Comparing melting curves for library at each round after PCR amplification gives qualitative information on convergence, as evidenced by an increase in higher-Tₘ species present in the library, presumably fully complementary duplexes. In the intermediate round libraries, there was a mix of higher and lower Tm species, whereas after 18 rounds of selection the library was composed of all high-Tm species indicating convergence to a small number of sequences.

Cloning of selected library-After rounds 13, 15, 17, and 18, 1 μL recovered template was amplified with hotstart Taq polymerase in a 100 μL PCR reaction as follows: 5 μL 10× standard Taq buffer, 1 μL 10 μM unmod-FP and 10 μM unmod-RP primers, 1 μL 10 mM DNTPs and 2.5 units of hot-start Taq polymerase (NEB: M0495S). 11 cycles of PCR were performed followed by incubation at 72° C. for 30 minutes to ensure optimal incorporation of overhanging adenosine nucleotides at the 3′ ends of both strands. A TOPO TA cloning kit was then used to clone the library according to manufacturer's instructions. Plasmid was transformed into competent XL-10 cells, plated and 24× colonies were picked into LB broth and the plasmid was isolated and Sanger sequenced. Of the 4 rounds selected, 6 different families of sequences were identified and labeled as follows, for example 13F1: Round 13 family 1 (highest frequency family in the recovered sequences obtained in round 13).

Selected clones-Sequence identifiers are shown below. Random Region (T's in the sequence correspond to positions where Man₉ moieties are located when the clone is prepared for binding assays) is shown in bold text. The forward primer is shown in underlined text. The reverse primer is shown in italicized text. The double-underline region is capture annealing/ssSpacer region. Sequences of clones studied in binding assays (+) strand, in 5′-3′ orientation

| Clone | | SEQ ID |
|---|---|---|
| Name | Sequence | NO: |
| 13F1 | CAACTGTAATACGACTCACTATAGGAGAGAAGGAGGGAAGAGAGAAAAAAAAA | 21 |
| | GGGTGTAGCCGTAAAGACCGTCAACAAGCGACACAAAGCCCGG | |
| 13F2 | CAACTGTAATACGACTCACTATAGGAGAGAAGGAAAGAGAGAAAAAAAAAACG | 22 |
| | CAAAGCGCCGCCATCGGATGCCAAGCGACACAAAGCCCGG | |
| 15F1 | CAACTGTAATACGACTCACTATAGGAGAGAAGGAGGGAAGAGAGAAAAAATCG | 23 |
| | ACGGAACAGGGGCCCCCTGTGGAAAGCGACACAAAGCCCGG | |
| 15F3 | CAACTGTAATACGACTCACTATAGGAGAGAAGGAGGGAAGAGAGAAAAAAAAC | 24 |
| | ATAGGGAACCCAGGTGATTGGGGCAAGCGACACAAAGCCCGG | |
| 17F3* | CAACTGTAATACGACTCACTATAGGAGACTGTCCCAACGGCAATGTGGCTCAC | 25 |
| | AAGCGACACAAAGCCCGG | |
| 18F4* | CAACTGTAATACGACTCACTATAGGAGACAGGATTGACACCGACCACTTGCCC | 26 |
| | AAGCGACACAAAGCCCGG | |

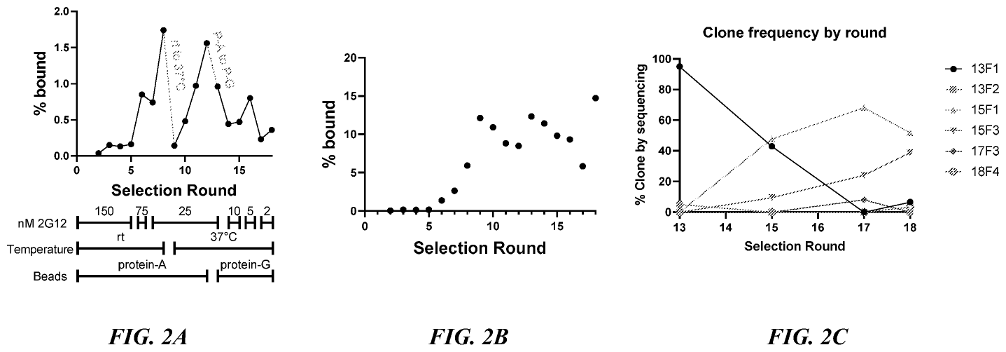

The overall distribution of these families in the colonies sequenced for each round is represented in main text (FIG. 2C). Both 17F3 and 18F4 sequences did not contain the double-underlined region representing the capture annealing sequence/ss-linker in the sequencing data, but nonetheless were tested in binding studies as truncated clones. Neither of these clones displayed any significant binding.

### Binding Studies

Preparation of selected clones for qPCR binding assay—For qPCR binding study, full glyco-RNA-DNA constructs presenting individual clones were prepared. Briefly, PCR of recovered purified plasmid with phosphate forward primer and IsodC reverse primer (as previously described) was performed to obtain individual phosphate/double stranded template. PCR reactions were cleaned up using DNA clean and concentrators (Zymo Research-D4029) according to manufacturer's protocol. Purified templates were then rendered single-stranded by digestion of phosphate-strand with lambda exonuclease, followed by desalting into water. 6 pmol purified clone template was built into full construct as previously described in the selection step.

qPCR binding study—To determine the K_{D} of individual clones, an individual sequence glyco-RNA/DNA hybrid (0.25 nM) was incubated with various concentrations of 2G12 (0, 1, 3, 10, 30, 100, 300 nM) for 30 minutes at room temperature. All binding assays were performed in duplicate. Bound construct was recovered by capturing 2G12 on Protein G Dynabeads (0.75 mg) by incubating for 15 minutes. Beads were washed (3×200 μL) to remove the non-bound constructs followed by analysis via qPCR. Similar to the selection, elution was performed by incubating the beads at room temperature in 3.5M MgCl₂ (50 uL) for 5 minutes; the supernatant was then buffer exchanged 2× with BE using Centri-spin 20 microcentrifuge columns. 1 μL of eluted construct was then analyzed in iTaq Universal SYBR Green Supermix (Biorad: 1725121) with 1 UM phosphate forward primer and IsodC reverse primer. qPCR mixture was analyzed using the same settings previously described for the selection.

Preparation of selected clones for filter binding assay—For binding studies, PCR of recovered purified plasmid with biotin forward and isodC reverse primers (as previously described) was performed to obtain individual biotinylated/double stranded template for facile removal post transcription. 1.5 pmol purified clone template was transcribed by adding 5 μL R&DNA T7 polymerase 5× reaction buffer, 2.5 μL 100 mM DTT, 0.5 μL 10 mM modified NTP mix (5-ethynyl-2′-fluoro-dUTP, 2′-fluoro-dCTP, rATP, rGTP) followed by 37.5 units of R&DNA T7 polymerase and heated to 37° C. for 60 minutes. Modified RNA was then functionalized with Man₉-cy-N₃ as previously described (CuAAC), buffer exchanged 2× into BE and incubated with 0.25 mg streptavidin magnetic beads to remove the double stranded template.

Dephosphorylation/Radiolabeling-RNA was then 5′-dephosphorylated by incubation with recombinant Shrimp Alkaline Phosphatase (rSAP) according to manufacturer's protocol then buffer exchanged into BE. The glycosylated and dephosphorylated ssRNA was then radioactively phosphorylated using polynucleotide kinase and ATP (γ-³²P) according to manufacturer's instructions. The desalted radiolabeled glycosylated aptamer was then used in the filter binding assay described below.

Filter-binding-Filter binding buffer (20 mM Tris pH 7.5, 150 mM NaCl, 2 mM MgSO₄, 50 μg/mL BSA) was prepared freshly and filtered through 0.2 μM syringe filter. 2G12 dilutions of 1000 nM, 250 nM, 62.5 nM, 15.6 nM, 3.9 nM, 0.98 nM, 0.24 nM and 0 nM were used in the filter binding assays. All binding assays were performed in duplicate.

Sufficient radiolabeled RNA (enough to produce an adequate radiogram after 90-minute exposure, generally 10-50 fmol) was diluted to 45 μL with filter binding buffer+BSA. 5 μL of the radiolabeled and diluted aptamer was added to a 50 μL aliquot of the antibody. After binding for 1 hr, the solution was vacuum filtered through a nitrocellulose/PVDF membrane sandwich and the radioactivity in each membrane was quantified by exposure to a phosphor screen followed by phosphor imaging. The data were fit to F_{bound}=(Fₘₐₓ[2G12])/K_{D}+[2G12]).

Nitrocellulose was soaked in binding buffer (NO salt) prior to the filter binding assay. PVDF was exposed to methanol for 10 minutes prior to extensive washing with H2O and soaking in binding buffer (NO salt) prior to the filter binding assay.

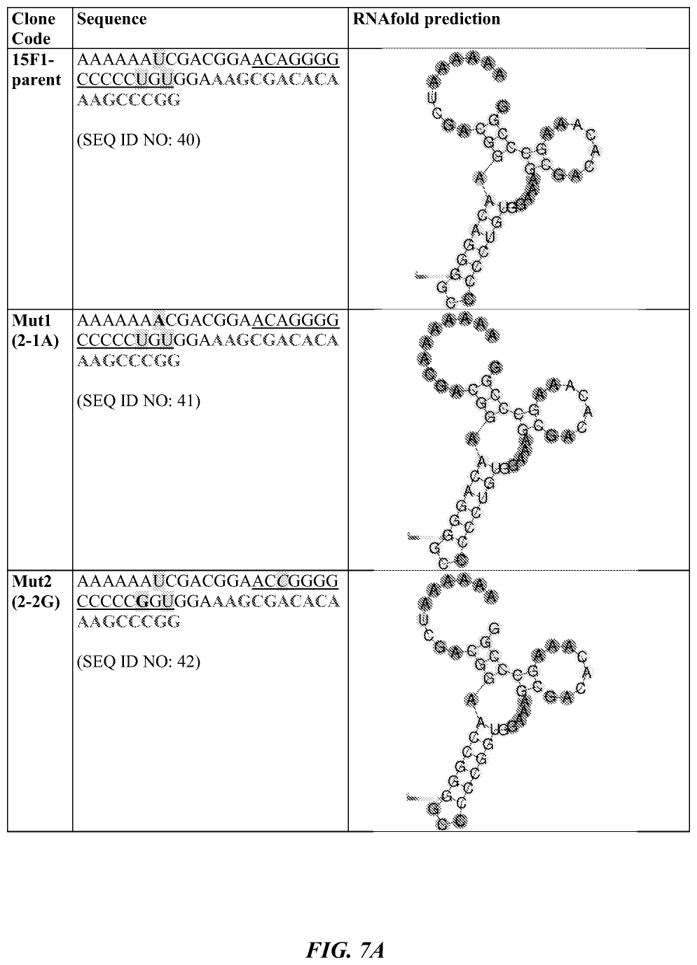

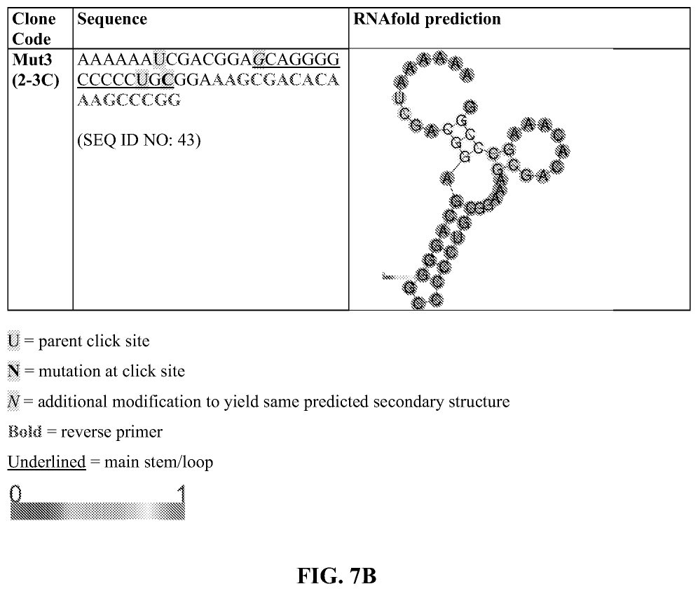

15F1 Mutants—To determine which Man₉ moieties are responsible for the observed 40 nM binding of 15F1, mutants were ordered from IDT replacing each T with another base that removed the click site as well as minimized predicted folding change (RNAfold) (Lorenz et al., Algorithms for Molecular Biology, 6 (1): 26 (2011), which is hereby incorporated by reference in its entirety) from the parent 3-click 15F1 aptamer. Due to the clone containing higher G/C, simple transition mutation did not always produce similar predicted fold to the parent clone so transversion mutations were also used for some mutants. These mutants are shown in FIGS. 7A-B, where (0-1)=RNAfold “base-pairing probability” (Lorenz et al., Algorithms for Molecular Biology, 6 (1): 26 (2011), which is hereby incorporated by reference in its entirety). For bases that are paired in the predicted structure, the color denotes the probability of being paired, and for unpaired bases in the predicted structure, color denotes the probability of being unpaired as described in FIGS. 7A-B.

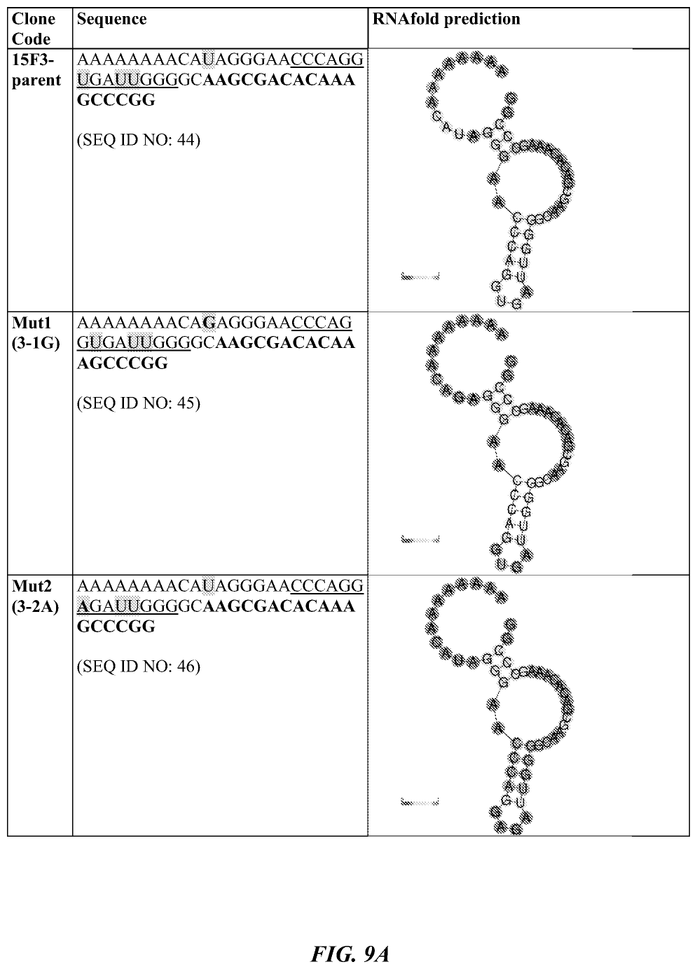

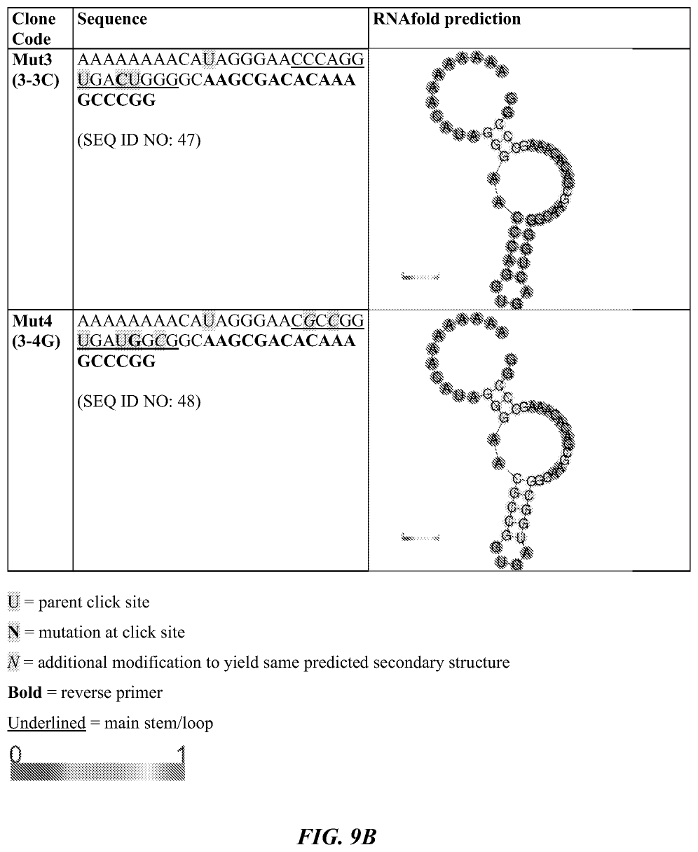

15F3 Mutants are shown in FIGS. 9A-B where (0-1)=RNAfold “base-pairing probability” (Lorenz et al., Algorithms for Molecular Biology, 6 (1): 26 (2011), which is hereby incorporated by reference in its entirety). For bases that are paired in the predicted structure, the color denotes the probability of being paired, and for unpaired bases in the predicted structure, color denotes the probability of being unpaired as shown in FIGS. 9A-B.

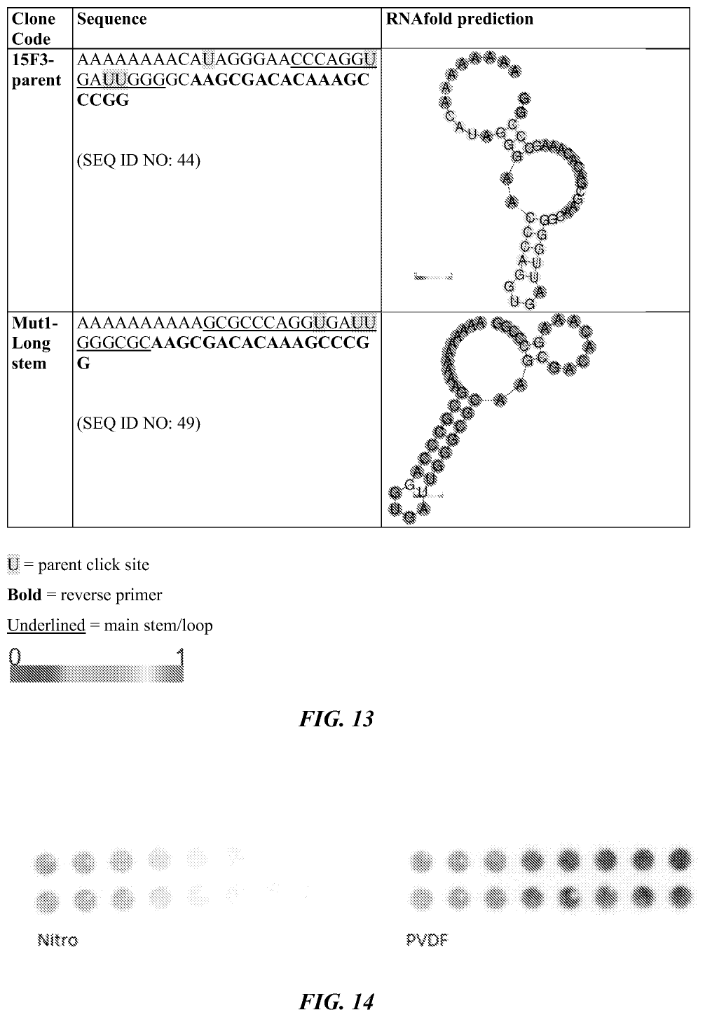

15F3-M1 modification with long stem are shown in FIG. 13. (0-1)=RNAfold “base-pairing probability” (Lorenz et al., Algorithms for Molecular Biology, 6 (1): 26 (2011), which is hereby incorporated by reference in its entirety). For bases that are paired in the predicted structure, the color denotes the probability of being paired, and for unpaired bases in the predicted structure, color denotes the probability of being unpaired as shown in FIG. 13. The stem of 15F3-M1 was stabilized by extending it by 3 G-C base pairs as well as removed extraneous sequence that contributed to multiple other predicted folds ensuring clone prepared would contain the predicted GNRA tetraloop stabilized by a longer stem region.

### General Synthesis Methods (2′F-EdUTP Synthesis)

All commercial reagents were used as provided unless otherwise indicated. Trimethyl phosphate and tributylamine were refluxed over CaH₂ and vacuum distilled into Schlenk flasks immediately before use. Phosphorus (V) oxychloride was refluxed, and vacuum distilled into Schlenk flask immediately before use. Triethylamine, N,N-diisopropylethylamine and pyridine were freshly distilled from CaH₂ at atmospheric pressure. Unless otherwise noted, all reactions were performed under N₂ atmosphere. Anhydrous DMF and acetonitrile were purchased from EMD as Dri-Solv bottles. Triethylammonium bicarbonate buffer was prepared by bubbling CO2 into a suspension of 138 mL triethylamine in 700 mL water until a homogenous solution at pH 8.0 was obtained. The solution was then diluted to 1 L with water. ¹H NMR resonances are reported in ppm 8 downfield of an external TMSP standard (0.00 ppm). ³¹P NMR resonances are referenced to an external phosphoric acid standard (0.0 ppm), and 19F NMR resonances are referenced to an external trifluoroacetic acid standard (−76.55 ppm).

### Example 1—Synthesis of 5-ethynyl-2′-fluoro-2′-deoxyuridine-5′-triphosphate (1)

5-ethynyl-2′-fluoro-2′-deoxyuridine-5′-triphosphate (1) was prepared for subsequent use during the Capture-SELMA procedure described in Example 2 beginning with 2′-fluoro-2′-deoxyuridine (2) as the starting material.

Using 2′-fluoro-2′-deoxyuridine (2) as the starting material, 5-iodo-2′-fluoro-2′-deoxyuridine (3) was prepared according to literature protocol, except that DMF instead of ionic liquid was used as solvent. Product spectra match those reported in the literature (Kumar et al., Synthesis, 2009:3957-3962 (2009), which is hereby incorporated by reference in its entirety).

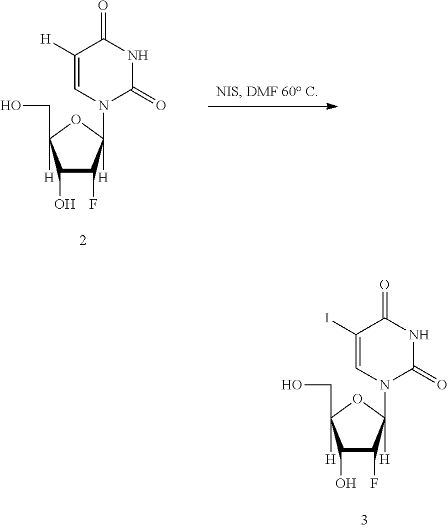

Next, 5-TES-ethynyl-2′-fluoro-2′-deoxyuridine (4) was prepared using 5-iodo-2′-fluoro-2′-deoxyuridine (3) from the preceding step. 5-iodo-2′-fluoro-2′-deoxyuridine (3) (400 mg, 1.08 mmol) was dissolved in freshly distilled Et₃N (32 mL) and anhydrous acetonitrile (32 mL). Pd(PPh₃)₂Cl₂ (16 mg, 0.023 mmol, 2.1 mol %) and CuI (16 mg, 0.086 mmol, 7.8 mol %) were added, followed by dropwise addition of triethylsilylacetylene (0.482 mL, 3.41 mmol, 3.17 equiv.). The reaction was heated to 60° C. and monitored by TLC (17:3, DCM:methanol) for the consumption of starting nucleoside. The reaction was complete at 60 minutes and concentrated in vacuo. Crude residue was dissolved in 99:1 DCM:methanol, purified by silica column (12″×1.25″ diameter, mobile phase: 19:1 DCM:methanol). Fractions containing product were combined and concentrated in vacuo to afford the product as an off-white powder (356 mg, 86%).

The intermediate product (4) was confirmed by ¹H NMR, ¹³C NMR, and ¹⁹F NMR as follows: ¹H NMR (400 MHZ, CD₃OD) δ 8.44 (s, 1H), 5.98 (br d, J=16.7 Hz, 1H), 5.02 (dd, J=52.9, 4.3 Hz, 1H), 4.33 (ddd, J=21.7, 8.0, 4.3 Hz, 1H), 4.09-3.94 (m, 2H), 3.77 (dd, J=12.4, 2.3 Hz, 1H), 1.03 (t, J=7.9 Hz, 9H), 0.65 (q, J=7.9 Hz, 6H); ¹³C NMR (101 MHZ, CD₃OD) δ (including three doublets due to J_{CF}) 164.07, 151.00, 145.79, 100.70, 98.49, 96.62, 96.03, 94.17, 90.12, 89.77, 84.69, 69.04, 68.88, 60.42, 7.76, 5.20; and ¹⁹F NMR (376 MHZ, CD₃OD) δ −204.58 (ddd, J=52.9, 21.7, 16.7 Hz).

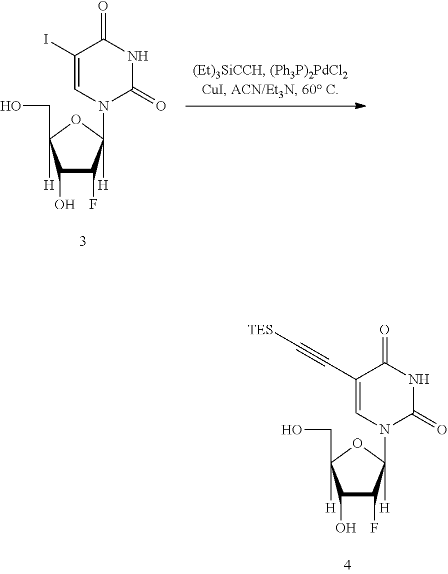

Next, 5-TES-ethynyl-2′-fluoro-2′-deoxyuridine-5′-triphosphate (5) was prepared using 5-TES-ethynyl-2′-fluoro-2′-deoxyuridine (4) from the preceding step. 5-TES-ethynyl-2′-fluoro-2′-deoxyuridine (4) (53 mg, 0.14 mmol) was dried azeotropically with pyridine (the material was dissolved in pyridine, frozen with liquid N₂, concentrated to dryness on the high vac and repeated 4 times) and left over P₂O₅ in vacuo overnight. Proton sponge (189.1 mg, 0.882 mmol) was added to dried nucleoside, under N₂, followed by the addition of trimethyl phosphate (2.75 mL). Solution was stirred 60 minutes room temperature, before being placed in a salt/ice bath maintained at −15° C. Phosphorus (V) oxychloride (55 μL, 0.593 mmol) was added to the cooled solution dropwise over 2 minutes, and the reaction was monitored by both TLC and LC-MS. To monitor, a ˜1 μL aliquot was quenched in 2 drops of 0.2M triethylammonium bicarbonate (TEAB) buffer, pH 8.0, and further diluted with water for LC-MS analysis. At 50 minutes, when starting nucleoside was fully consumed by LC-MS, a premixed solution of tributylammonium pyrophosphate (620 mg, 1.13 mmol) and freshly distilled tributylamine (147 μL, 0.62 mmol) in dry DMF (647 μL) was added to monophosphate reaction over X minutes. At 60 minutes, reaction was no longer progressing, so reaction was quenched by adding solution dropwise to a cooled solution of 0.2M TEAB (16 mL) and stirring on ice for 45 minutes. This solution was concentrated in vacuo, redissolved in water (3 mL) and passed through a column of Dowex-H⁺ 50WX8 (Na⁺ form). Additional water was added to the column until nucleotide fully eluted, as monitored by X (6×8 mL fractions). Fractions containing triphosphate were pooled and lyophilized. Crude material was purified by preparative HILIC-HPLC (19×250 mm Waters X-Bridge prep HILIC column, 5 μm OBD) monitored at 315 nm, with a gradient starting at 100% B for 10 minutes, followed by 100-70% B over 45 min, where B was 90% MeCN+10% 1M TEAB pH 8 and A was 50% MeCN+10% 1M TEAB pH 8.0. Pure triphosphate eluted at 46.4 minutes, was lyophilized, and passed once more through a small column of Dowex-H⁺ 50WX8 (Na⁺ form) to exchange triethylammonium ions for sodium. Fractions containing triphosphate were pooled and lyophilized to afford pure product as a white powder (20.0 mg, 22%).

The intermediate product (5) was confirmed by ¹H NMR, ¹³C NMR, and ¹⁹F NMR as follows: ¹H NMR (400 MHZ, D₂O) δ 7.87 (s, 1H), 5.78 (dd, J=20.2, 1.6 Hz, 1H), 5.11 (ddd, J=52.8, 5.1, 1.6 Hz, 1H), 4.35 (ddd, J=20.2, 7.8, 5.1 Hz, 1H), 4.22-4.03 (m, 3H), 0.84 (t, J=7.9 Hz 9H), 0.53 (q, J=7.9 Hz, 6H). ¹⁹F NMR (376 MHZ, D₂O) δ −201.01 (ddd, J=52.9, 20.1, 20.1 Hz). ³¹P NMR (162 MHZ, D₂O) δ −9.64 (d, J=19.8 1P), −10.59 (dd, J=19.8, 4.6 Hz, 1P), −22.21 (app t, J=19.8 Hz, 1P).

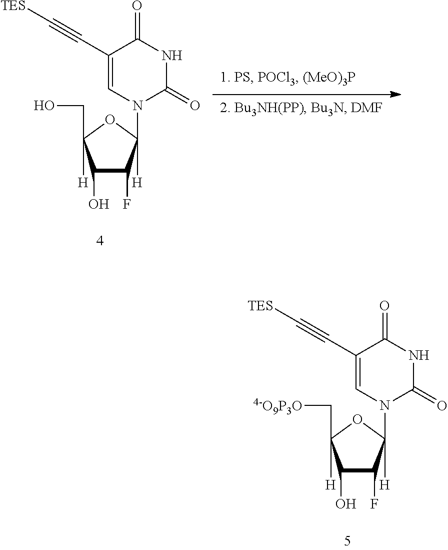

Finally, 5-ethynyl-2′-fluoro-2′-deoxyuridine-5′-triphosphate (1) was prepared from 5-TES-ethynyl-2′-fluoro-2′-deoxyuridine-5′-triphosphate (5) obtained in the preceding step. 5-TES-ethynyl-2′-fluoro-2′-deoxyuridine-5′-triphosphate (5) (6.0 mg, 9.0 umol) was transferred to a 4 mL vial with a magnetic stir bar. Protected triphosphate was dissolved in dry acetonitrile (673 μL), followed by 224 μL tetrabutylammonium fluoride (1M in THF). Reaction was stirred at room temperature and monitored by LCMS for complete deprotection of the alkyne. At 18 hr the reaction was complete, and product was separated from excess TBAF by passing through a pipet column of DEAE-Sephadex (Cl form) previously hydrated in water. Product was eluted from the column by increasing concentrations (50 mM-1M) of TEAB pH 8.0, with deprotected triphosphate eluting between 400-500 mM. Pure fractions, confirmed by LCMS, were pooled and concentrated in vacuo then passed through a small column of Dowex-H⁺ 50WX8 (Na⁺ form) to exchange triethylammonium ions for sodium. Fractions containing deprotected triphosphate were detected by TLC and LCMS, pooled and lyophilized to afford the pure product as a white powder (4.5 mg, 91%).

The final product (1) was confirmed by ¹H NMR, ¹⁹F NMR, and ³¹P NMR as follows: ¹H NMR (400 MHZ, D₂O) δ 8.21 (s, 1H), 6.07 (br d, J=18.1 Hz, 1H), 5.20 (dd, J=52.0, 4.6 Hz, 1H), 4.55 (ddd, J=22.6, 8.4, 4.6 Hz, 1H), 4.42-4.27 (m, 3H), 3.60 (s, 1H); ¹⁹F NMR (376 MHz, D₂O) δ −203.27 (ddd, J=52.0, 22.6, 18.1 Hz); ³¹P NMR (162 MHZ, D₂O) δ −7.50 (m, 1P), −10.68 (d, J=20.0 Hz, 1P), −21.80 (app t, J=20.0 Hz, 1P).

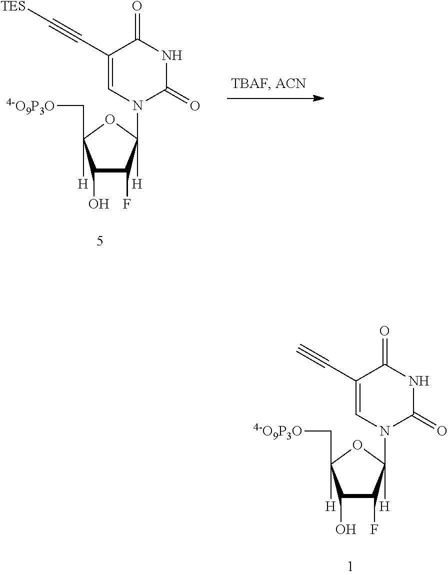

The final product, 5-ethynyl-2′-fluoro-2′-deoxyuridine-5′-triphosphate (1), was used in Example 2 to prepare and selected 2′-fluoro-modified, RNA-supported Carbohydrate Clusters

### Example 2—Directed Evolution of 2′-Fluoro-Modified, RNA-Supported Carbohydrate Clusters that Bind Tightly to HIV Antibody 2G12

A transcription was carried out in the presence of ribopurines, but 2′-deoxy-2′-fluoropyrimidines—where uridine is replaced with 2′-deoxy-2′-fluoro (5-ethynyl) uridine—was used for CuAAC (Rostovtsev et al., Angew. Chem. Int. Ed., 41 (14): 2596-2599 (2002), which is hereby incorporated by reference in its entirety) attachment of glycan azides (Gordon et al., ACS Chem. Biol., 14 (12): 2652-2662 (2019); Bachem et al., Angew. Chem. Int. Ed., 59 (47): 21016-21022 (2020); Novoa et al., Beilstein J. Org. Chem. 11:707-719 (2015); and Gierlich et al., Chem. Eur. J., 13 (34): 9486-9494 (2007), which are hereby incorporated by reference in their entirety). The resulting glycosylated F-RNA library (Form G) then undergoes selection for binding to the target and the bound fraction is amplified by PCR with a biotinylated forward primer (Form H). The biotinylated strand is then removed with streptavidin beads, regenerating Form A of the library to begin the next round of selection.

Several features of the oligonucleotide design were important for optimal library function. First, to avoid early transcript termination in the presence of fluoropyrimidines (Zhu et al., Nucleic Acids Res., 43 (14): e94 (2015), which is hereby incorporated by reference in its entirety), the beginning of the RNA sequence (blue) was designed to contain only natural purine bases. Additionally, this sequence, which must anneal to the capture strand, was designed to avoid formation of G quadruplexes (Huppert et al., FEBS J., 277 (17): 3452-8 (2010) and Bochman et al., Nat. Rev. Genet., 13 (11): 770-80 (2012), which are hereby incorporated by reference in their entirety) or stable secondary folds that could interfere with efficient hybridization of the RNA transcript to the capture strand (see FIGS. 5A-5B for illustration of library construction). A selection was attempted prior to optimization of this sequence, which failed to yield strong enrichment or tight binders.

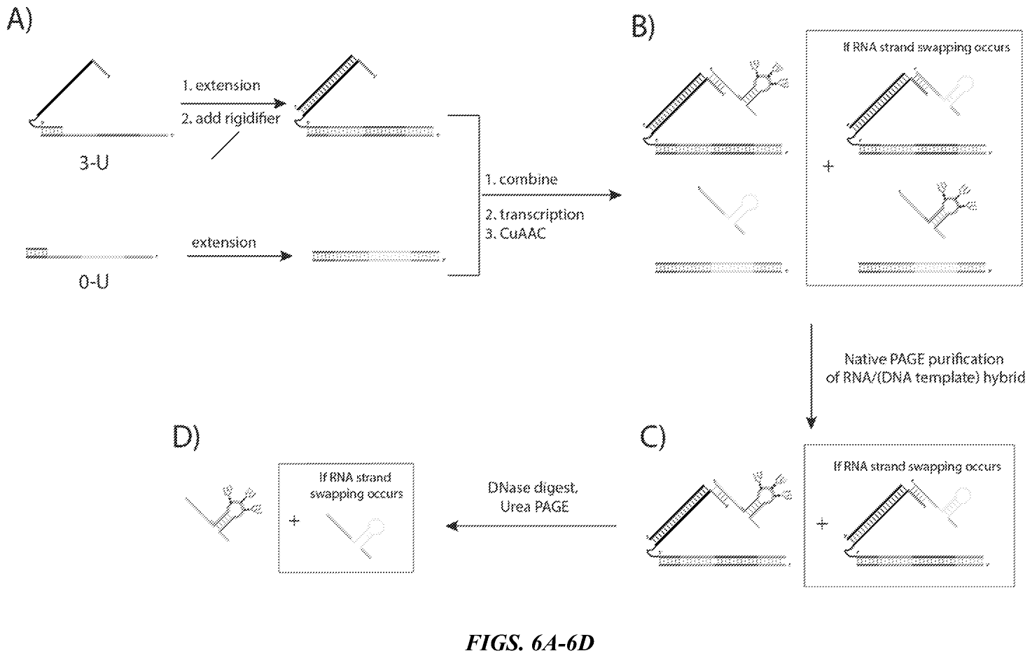

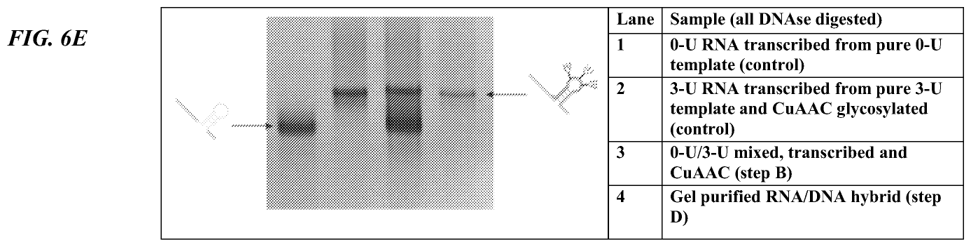

A control experiment verified that the captured RNA strand did not significantly exchange with free RNA transcripts in solution (FIGS. 6A-6E). A poly(A) spacer (orange) was included to distance the capture annealed region from the random region (red) bearing the glycans. The required ethynyl fluorinated uridine triphosphate (1) was prepared as described in Example 1, and was readily incorporated by R&D polymerase into the transcription product. Denaturing and native polyacrylamide gels exhibited bands of the expected size for each stage of the selection cycle.

With an optimized protocol in hand for generating the library, selection was commenced for 2G12 binders. For the first round, a 20 pmol library (˜1.2×10¹³ library members) was incubated with 150 nM 2G12, and the bound complexes were captured on Protein A magnetic beads. The bead-bound library fraction was eluted by 3.5 M MgCl₂ disruption of the antibody/protein-A interaction and quantified by qPCR (FIG. 2A). Although successive rounds of selection included more stringent conditions (lower antibody concentration, higher temperature, increased washes, see FIG. 2A and Table 3), a small parallel selection was run under the original conditions at each round to assess enrichment (FIG. 2B). Prior to each selection with target, the library was incubated with empty beads and the supernatant was transferred to another tube to select against bead binders or plastic binders.

When qPCR indicated increases in library recovery, stringency was increased by lowering the 2G12 target concentration (eventually down to 2 nM), increasing the number of wash cycles and volume for bead-bound library, increasing selection temperature from room temperature to 37° C., or switching from Protein A to Protein G beads. Recovery increased dramatically in the first eight rounds (FIG. 2B) from <0.1% to ˜10%. The increase of selection temperature in round 9 and the switch to Protein G beads in round 13 each resulted in temporary decreases in library recovery (FIG. 2A), which typically increased again after multiple rounds under the same conditions.

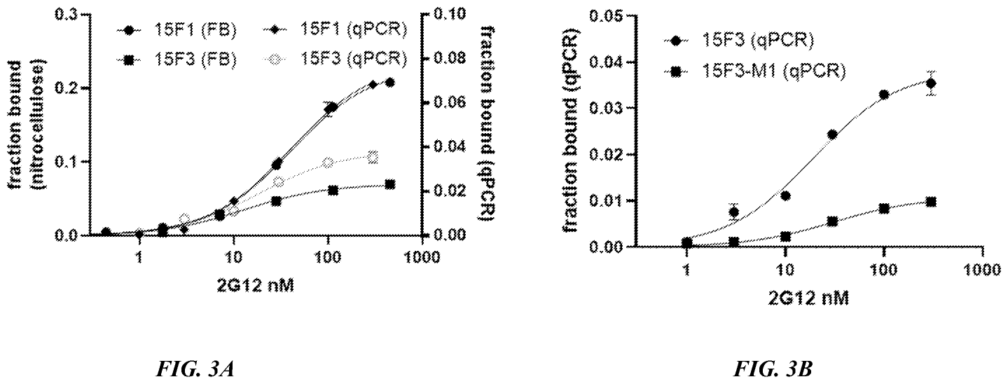

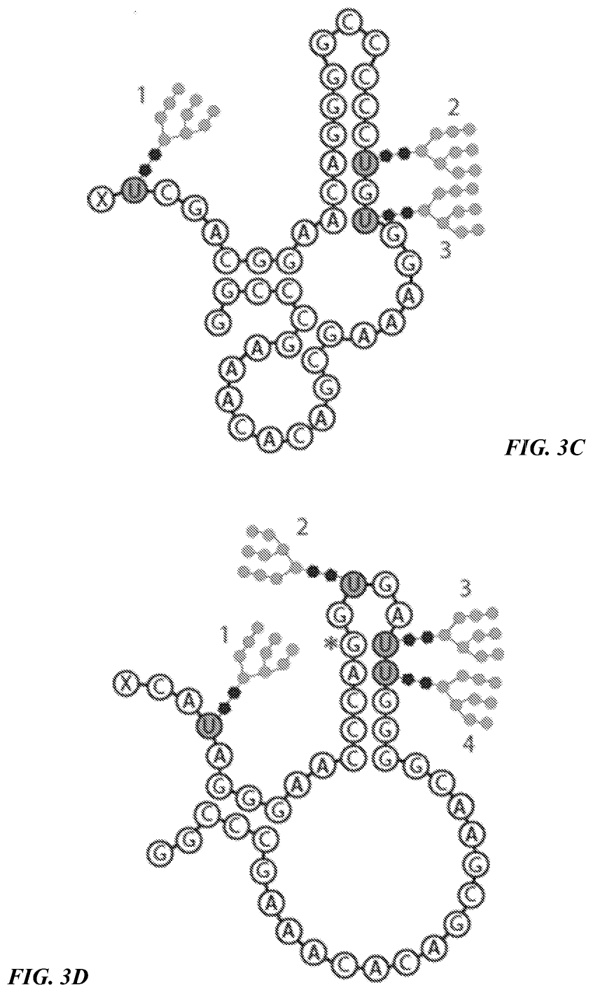

Throughout selection, the number of glycans per library member, as assessed by PAGE of DNAse-digested library, narrowed from a broad distribution to 2-4 glycans/clone, and the melting behavior of the library DNA visibly shifted to a higher temperature distribution. 36 clones from rounds 13, 15, 17 and 18 were sequenced and their 2G12 binding behavior assessed (FIGS. 2C and 3A-3D; Table 4). Nitrocellulose filter binding assays (Rio, D. C., Cold Spring Harb. Protoc. (2012), which is hereby incorporated by reference in its entirety) were conducted with glycosylated F-RNA transcripts, whereas in an alternative assay, clones were produced as glycosylated F-RNA/DNA hybrids (Form G) and qPCR was used to quantify 2G12-bound complexes captured on protein A beads.

Among the major clones observed in the final rounds, 15F1 and 15F3 became most dominant after rounds 17 and 18. They bound (FIG. 3A; Table 4) with nanomolar affinity to 2G12 (K_{D}s of 40±6 and 13±2 nM, respectively) in the filter binding assay, and those results agreed well with the qPCR binding assay results (K_{D}s of 40±4 and 19±3 nM, respectively). In the predicted secondary fold (RNAfold) (Lorenz et al., 2.0. Algorithms Mol. Biol., 6 (1): 26 (2011), which is hereby incorporated by reference in its entirety) of both clones, the reverse primer constant sequence hybridizes with three bases at the 5′-end of the aptamer where one glycan is present, whereas the remaining 2-3 glycans are on a stem-loop feature (FIGS. 3C and 3D).

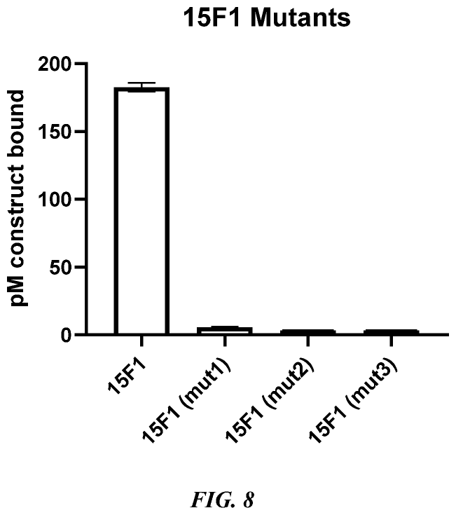

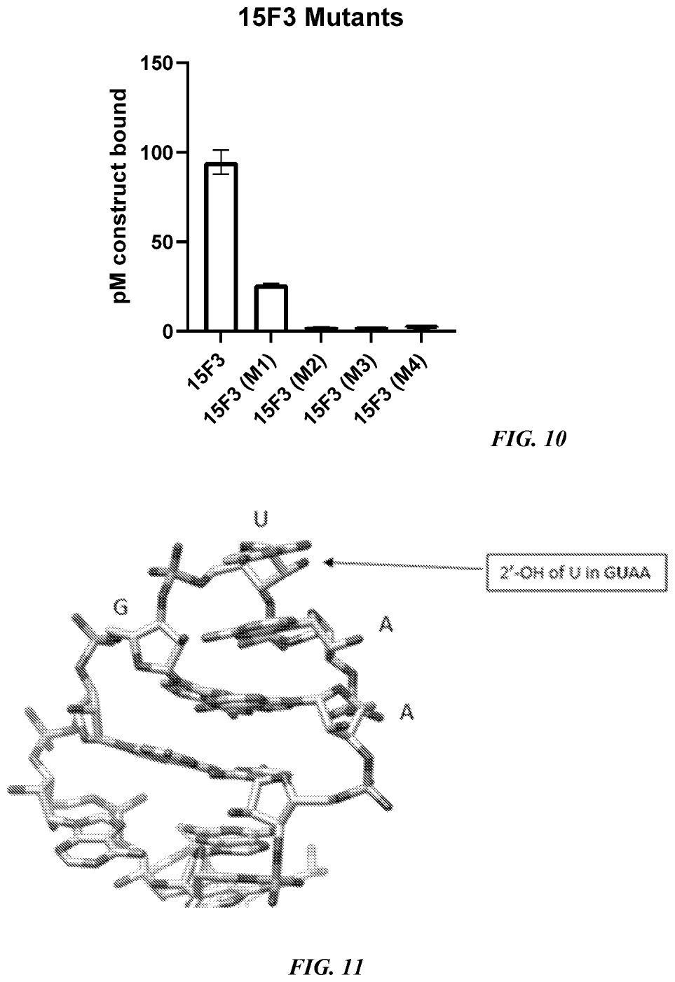

To further understand the structure and glycan-dependency of binding of the two clones that displayed nanomolar binding, single U->X transition or transversion point mutations were tested to remove each possible glycosylation site (three for 15F1 and four for 15F3) while the complementary base in the predicted stem structure was mutated to retain the predicted fold (FIGS. 3E, 7, and 9). The qPCR binding method was used to test each mutant at 0 nM and 300 nM 2G12 (FIGS. 8 and 10). Only 15F3-mutant 1 (“15F3-M1”) displayed any binding to 300 nM 2G12, suggesting that three glycosylation sites are critical in both clones, with only the fourth glycan of 15F3 being superfluous for binding. A full concentration series was performed to measure the K_{D} of 15F3-M1, revealing a modest affinity reduction (31±4 nM versus 19±3 nM) and a significant decrease in maximum concentration bound, compared with the parent clone (FIG. 3B; Table 4).

TABLE 4
Binding Data and Sequences

| | | | Filter | | qPCR Bead | |
|---|---|---|---|---|---|---|
| Clone | | | Binding | | Binding | |
| ID | Glycans | Sequence | K_{d} (nM) | % Fbₘₐₓ | K_{d} (nM) | %Fbₘₐₓ |
| 15F1 | 3 | UCGACGGAACAGGGGCCCCCUGUGGA | 40 ± 6 | 23 ± 1 | 40 ± 4 | 7.9 ± 0.3 |
| | | (SEQ ID NO: 27) | | | | |
| 15F1-M1 | 2 | ACGACGGAACAGGGGCCCCCUGUGGA | nd | nd | nb | nb |
| | | (SEQ ID NO: 28) | | | | |
| 15F1-M2 | 2 | UCGACGGAACCGGGGCCCCCGGUGGA | nd | nd | nb | nb |
| | | (SEQ ID NO: 29) | | | | |
| 15F1-M3 | 2 | UCGACGGAGCAGGGGCCCCCUGCGGA | nd | nd | nb | nb |
| | | (SEQ ID NO: 30) | | | | |
| 15F3 | 4 | CAUAGGGAACCCAGGUGAUUGGGGC | 13 ± 2 | 7.0 ± 0.3 | 19 ± 3 | 3.8 ± 0.2 |
| | | (SEQ ID NO: 31) | | | | |
| 15F3-M1 | 3 | CAGAGGGAACCCAGGUGAUUGGGGC | nd | nd | 31 ± 4 | 1.1 ± 0.1 |
| | | (SEQ ID NO: 32) | | | | |
| 15F3-M2 | 3 | CAUAGGGAACCCAGGAGAUUGGGGC | nd | nd | nb | nb |
| | | (SEQ ID NO: 33) | | | | |
| 15F3-M3 | 3 | CAUAGGGAACCCAGGUGACUGGGGC | nd | nd | nb | nb |
| | | (SEQ ID NO: 34) | | | | |
| 15F3-M4 | 3 | CAUAGGGAACGCCGGUGAUGGCGGC | nd | nd | nb | nb |
| | | (SEQ ID NO: 35) | | | | |

Bold U = 2′-deoxy-2′-fluoro(5-ethynyl)uridine glycosylation sites; italics = mutated glycosylation sites; underlining - auxiliary mutations that maintain stem stability after glycosylation site mutation; nd = not determined; nb = no binding at 300 nM 2G12.
Data were obtained in duplicate.
Reported errors are the standard error of the curve fit.

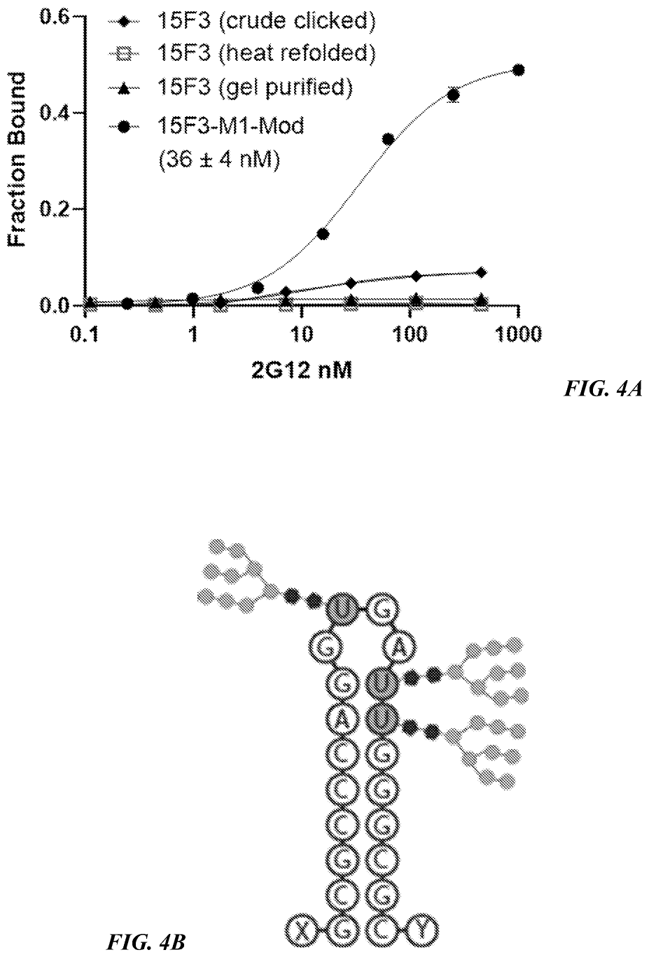

The observed Fbₘₐₓ values were low (<˜20%); to assess whether this was because of incompletely glycosylated aptamer, the fully glycosylated 15F1 and 15F3 F-RNA were purified by urea PAGE and repeated the nitrocellulose filter binding assay. Although glycoDNA aptamers from SELMA and natural RNA aptamers derived from the capture method exhibited excellent binding after PAGE purification (MacPherson et al., Angew. Chem. Int. Ed., 50 (47): 11238-11242 (2011); Temme et al., Chem. Eur. J., 19 (51): 17291-17295 (2013); Temme et al., J. Am. Chem. Soc., 136 (5): 1726-1729 (2014); and MacPherson et al., Chem. Comm., 53 (19): 2878-2881 (2017), which are hereby incorporated by reference in their entirety), in this case, fully glycosylated 15F1 and 15F3 exhibited minimal binding signal after PAGE purification (FIG. 4A). It was hypothesized that the active 15F1 and 15F3 aptamers might be in kinetically trapped folding states after transcription and/or glycosylation, which convert to thermodynamically favored but inactive states upon denaturing PAGE purification and refolding.

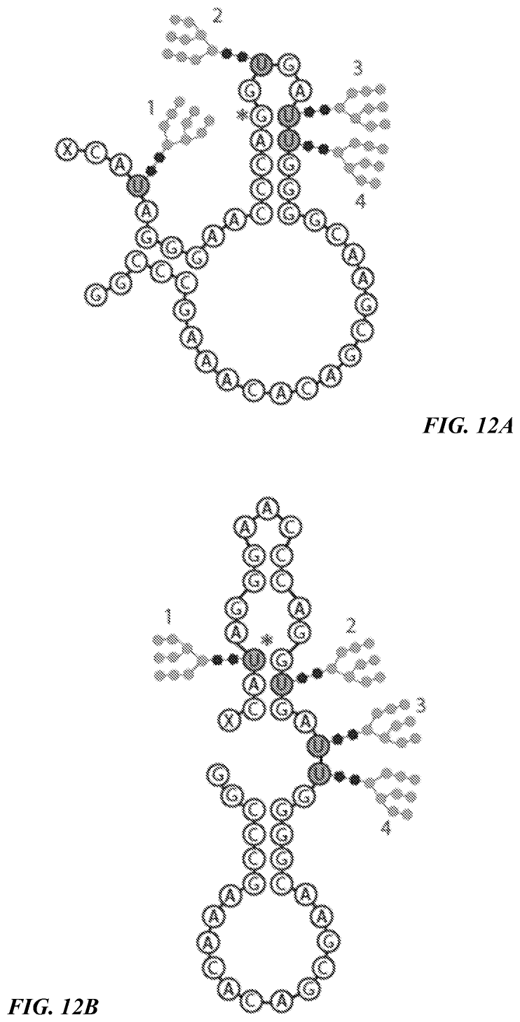

In the case of 15F3, further inspection of the predicted fold (FIG. 3D) suggested a route to stabilization of the structure. Three of the four glycans in 15F3 reside near the end of a stem or in a GNRA tetraloop (specifically GUGA), which is a common natural RNA folding motif (Batey et al., Angew. Chem. Int. Ed., 38 (16): 2326-2343 (1999), which is hereby incorporated by reference in its entirety). Although this fold has not known to have been reported in 2′-fluoro-pyrimidine aptamers, the major hydrogen bonding interactions that confer stability to the tetraloop are all between the purines at the first, third and fourth positions, with base stacking contributing to stability between the 2-4 bases in the loop in some variations (Cheong et al., eLS, pp. 1-6 (2015); Richardson, et al., Biochemistry, 58 (48): 4809-4820 (2019); and Correll et al., RNA 9 (3): 355-363 (2003), which are hereby incorporated by reference in their entirety). Therefore, the 2′-fluoro uridine could reasonably be accommodated in the canonical GNRA structure (FIG. 11). However, following attachment of the bulky Man₉ moieties to the loop and two stem positions, the structure might be kinetically stable in selection, but not retain enough thermodynamic stability to refold to the same structure after denaturing urea-gel purification. Based on mFold (Zuker, M., Nucleic Acids Res., 31 (13): 3406-3415 (2003), which is hereby incorporated by reference in its entirety) predictions, another possible fold with similar thermodynamic stability contains a completely different stem/loop not involving any of the glycosylation sites (FIG. 12).

To increase the stability of this potentially important stem tetraloop motif, the stem was extended (15F3-M1-Mod, see FIGS. 4B, 13 and 14). This modification necessitated the loss of the “non-essential” glycosylation site (see 15F3-M1). This long-stem variant was transcribed, click glycosylated, gel purified, then radiolabeled and refolded. In the nitrocellulose filter binding assay, strong binding (K_{D} 36±4 nM) is now observed after gel purification/refolding, and a significantly higher Fbₘₐₓ (51%) compared to the parent clone, which displayed no binding at all after gel purification (FIG. 4A). These data support the hypothesis that the active structure contains this stem-GNRA tetraloop, which is thermodynamically unstable in the original 15F3 aptamer.

It is worth noting that 15F3 and 15F3-M1-Mod, with K_{D}s of 13±2 and 36±4 nM respectively, exhibit 14,000- and 5000-fold binding enhancements relative to monovalent Man₉ (K_{D} 180 μM). Moreover, this affinity enhancement is about 17-50-fold greater than that achieved by unstructured glycan clusters of comparable multivalency, such as the trivalent Man₉ dendrimer described by Wong (Wang et al., PNAS, 105 (10): 3690 (2008), which is hereby incorporated by reference in its entirety).

In summary, Capture SELMA has been devised, a selection method for evolving glycan-modified 2′-fluoro-RNA carbohydrate cluster ligands capable of presenting oligomannose Man₉ glycans in a cluster that leads to very tight (mid- to low-nanomolar K_{D}) binding for the carbohydrate-binding antibody 2G12. This selection method is applicable to the discovery of F-RNA aptamers containing other modifications, appropriate for binding to diverse targets.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. All of the features described herein (including any accompanying claims, abstract and drawings), and/or all of the steps of any method or process so disclosed, may be combined with any of the above aspects in any combination, except combinations where at least some of such features and/or steps are mutually exclusive. Additionally, the recited order of processing elements or sequences, or the use of numbers, letters, or other designations therefore, is not intended to limit the claimed processes to any order except as may be specified in the claims. These alterations, improvements, and modifications are intended to be suggested hereby, and are within the spirit and scope of the invention. Accordingly, the invention is limited only by the following claims and equivalents thereto.

1. A method for selecting an RNA molecule that binds to a target molecule comprising:
providing a pool of oligonucleotide complexes that each comprise a ds-DNA molecule and an RNA molecule, the ds-DNA molecule comprising a first DNA strand at least partially annealed to a first region of the RNA molecule, whereby a second region of the RNA molecule is free to adopt a secondary structure;
exposing the pool to a target molecule and allowing the second region of the RNA to bind the target molecule; and
selecting from the pool one or more oligonucleotide complexes comprising an RNA molecule having the second region bound to the target molecule; wherein the RNA molecule comprises two to five modified nucleotides and/or modified ribosyl-phosphate groups within the second region, the first region of the RNA molecule comprises only natural bases and lacks a stable secondary structure, or both.

2. The method according to claim 1, wherein the first DNA strand comprises a 5′ capture region that is unpaired within the ds-DNA molecule and the RNA molecule comprises the first region at the 5′ end thereof and the second region extending from the first region to the 3′ end thereof, wherein the first region of the RNA molecule is complementary to and annealed to the 5′ capture region of the first DNA strand.

3. The method according to claim 2, wherein the annealed 5′ capture region and first region of the RNA molecule comprises a melting temperature of at least about 60° C.

4. The method according to claim 3, wherein the first DNA strand comprises a first portion that is annealed to a template strand, and a second portion that comprises the 5′ capture region and is tethered at the 3′ end thereof to the 5′ end of the first portion via a linker molecule.

5. The method according to claim 4, wherein the template strand comprises a random nucleotide sequence region in between first and second fixed nucleotide sequence regions respectively located 3′ and 5′ of the random nucleotide sequence, and the first fixed nucleotide sequence region comprises a poly-A sequence.

6. The method according to claim 1, wherein the modified nucleotides comprise an oligosaccharide linked to a 2′-fluoro-2′-deoxyuridinyl group at position 5 of the uridinyl group.

7. An isolated RNA molecule comprising from two to five modified nucleotides, wherein the modified nucleotides comprise an oligosaccharide linked to a 2′-fluoro-2′-deoxyuridinyl group at position 5 of the uridinyl group, wherein the RNA molecule binds to HIV neutralizing antibody 2G12 with an affinity (Kd) of about 40 nM or less.

8. The isolated RNA molecule according to claim 7, wherein the RNA comprises two to four modified nucleotides.

9. The isolated RNA molecule according to claim 7, wherein the oligosaccharide is a Man₉ group linked via a cyclohexyl-triazole linker to the uridinyl 5 position.

10. (canceled)

11. The isolated RNA molecule according to claim 7, wherein the RNA molecule binds to antibody 2G12 with an Fbmax of 20% of greater, or wherein the RNA molecule binds to antibody 2G12 with a Kd of 20 nM or less.

12. (canceled)

13. (canceled)

14. The isolated RNA molecule according to claim 7, wherein the RNA molecule comprises a stem-loop structure comprising two of the modified nucleotides at adjacent positions in the stem and one of the modified nucleotides in the loop.

15. The isolated RNA molecule according to claim 7, wherein the RNA molecule comprises the structure of 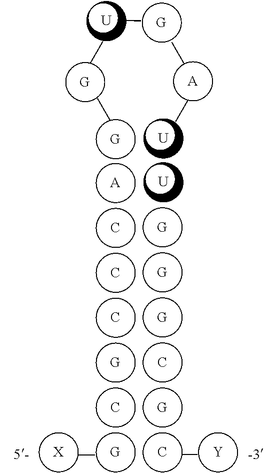
where X is optionally a poly-A sequence and Y is optionally AAGCGACACAAAGCCCGG-3′.

16. An immunogenic conjugate comprising the RNA molecule according to claim 7 covalently or non-covalently bound to an immunogenic carrier molecule.

17. The immunogenic conjugate according to claim 16, wherein the immunogenic carrier molecule is selected from the group consisting of bovine serum albumin, chicken egg ovalbumin, keyhole limpet hemocyanin, tetanus toxoid, diphtheria toxoid, thyroglobulin, a pneumococcal capsular polysaccharide, CRM 197, and a meningococcal outer membrane protein.

18. A pharmaceutical composition comprising a pharmaceutically acceptable carrier and the RNA molecule according to claim 7, or an immunogenic conjugate comprising the RNA molecule covalently or non-covalently bound to an immunogenic carrier molecule.

19. (canceled)

20. A method of inducing an immune response in an individual comprising:
administering to an individual the RNA molecule according to claim 7 or an immunogenic conjugate comprising the RNA molecule covalently or non-covalently bound to an immunogenic carrier molecule, wherein said administering is effective to induce an immune response against the oligonucleotide.

21. The method according to claim 20, wherein said administering is effective to induce a carbohydrate-binding, neutralizing antibody response.

22. (canceled)

23. (canceled)

24. (canceled)

25. (canceled)

26. (canceled)

27. A method of inhibiting HIV-1 infection or proliferation comprising:
administering to an individual the RNA molecule according to claim 7 or an immunogenic conjugate comprising the RNA molecule covalently or non-covalently bound to an immunogenic carrier molecule, wherein said administering is effective to induce a neutralizing immune response against HIV-1.

28. A method for detecting a neutralizing antibody in serum comprising:
providing the RNA molecule according to claim 7;
contacting the oligonucleotide with serum from an individual; and
detecting whether the oligonucleotide binds specifically to an antibody present in the serum, wherein said detecting is carried out using a label.


SEQUENCE LISTING (49 sequences)

SEQ ID NO: 1 - RNA, 20 nt, Artificial
  stem-loop structure
  misc_feature (10)..(10): n is a, c, g, or u
  misc_feature (13)..(14): n is a, c, g, or u
gcgcccaggnganngggcgc

SEQ ID NO: 2 - RNA, 18 nt, Artificial
  stem-loop structure
aagcgacacaaagcccgg

SEQ ID NO: 3 - DNA, 92 bp, Artificial
  Library Sequence
  misc_feature (19)..(43): N at positions 19-43 can be A, T, C, or G
ccgggctttgtgtcgcttnnnnnnnnnnnnnnnnnnnnnnnnntttttctctcttccctccttctctcctatagtgagtcgtattacagttg

SEQ ID NO: 4 - DNA, 119 bp, Artificial
  CapArm
ctctcttccctccttctctcctttttcaacaccacagaccagtatacccagaaatgacgcaagcatagacaaacgatttagacatgagtgcccgacacaacgaacaagcttttttttta

SEQ ID NO: 5 - DNA, 28 bp, Artificial
  CapArm
caactgtaatacgactcactataggaga

SEQ ID NO: 6 - DNA, 83 bp, Artificial
  Rigidifier
gcttgttcgttgtgtcgggcactcatgtctaaatcgtttgtctatgcttgcgtcatttctgggtatactggtctgtggtgttg

SEQ ID NO: 7 - DNA, 28 bp, Artificial
  Primer
caactgtaatacgactcactataggaga

SEQ ID NO: 8 - DNA, 18 bp, Artificial
  IsodC Rev primer
ccgggctttgtgtcgctt

SEQ ID NO: 9 - DNA, 96 bp, Artificial
  3-U primer
ccgggctttgtgtcgctttatccgtaggttgcaccgtgggtcttttttttttctctcttccctccttctctcctatagtgagtcgtattacagttg

SEQ ID NO: 10 - DNA, 96 bp, Artificial
  0-U primer
ccgggctttgtgtcgctttgtccgtgggttgcgccgcggccgctttttttttctctcttccctccttctctcctatagtgagtcgtattacagttg

SEQ ID NO: 11 - DNA, 94 bp, Artificial
  15F1-M1
ccgggctttgtgtcgctttccacagggggcccctgttccgtcgtttttttctctcttccctccttctctcctatagtgagtcgtattacagttg

SEQ ID NO: 12 - DNA, 94 bp, Artificial
  15F1-M2
ccgggctttgtgtcgctttccactgggggcccctgttccgtcgattttttctctcttccctccttctctcctatagtgagtcgtattacagttg

SEQ ID NO: 13 - DNA, 94 bp, Artificial
  15F1-M3
ccgggctttgtgtcgctttcctcagggggcccctgttccgtcgattttttctctcttccctccttctctcctatagtgagtcgtattacagttg

SEQ ID NO: 14 - DNA, 95 bp, Artificial
  15F3-M1
ccgggctttgtgtcgcttgccccaatcacctgggttccctctgttttttttctctcttccctccttctctcctatagtgagtcgtattacagttg

SEQ ID NO: 15 - DNA, 95 bp, Artificial
  15F3-M2
ccgggctttgtgtcgcttgccccaatctcctgggttccctatgttttttttctctcttccctccttctctcctatagtgagtcgtattacagttg

SEQ ID NO: 16 - DNA, 95 bp, Artificial
  15F3-M3
ccgggctttgtgtcgcttgccccagtcacctgggttccctatgttttttttctctcttccctccttctctcctatagtgagtcgtattacagttg

SEQ ID NO: 17 - DNA, 95 bp, Artificial
  15F3-M4
ccgggctttgtgtcgcttgccgccatcaccggcgttccctatgttttttttctctcttccctccttctctcctatagtgagtcgtattacagttg

SEQ ID NO: 18 - DNA, 92 bp, Artificial
  15F3-Mut-1-Mod
ccgggctttgtgtcgcttgcgcccaatcacctgggcgcttttttttttctctcttccctccttctctcctatagtgagtcgtattacagttg

SEQ ID NO: 19 - RNA, 25 nt, Artificial
  3-U Capture Arm Primer
agacccacggugcaaccuacggaua

SEQ ID NO: 20 - DNA, 25 bp, Artificial
  0-U Forward Primer
gcggccgcggcgcaacccacggaca

SEQ ID NO: 21 - DNA, 96 bp, Artificial
  13F1
caactgtaatacgactcactataggagagaaggagggaagagagaaaaaaaaagggtgtagccgtaaagaccgtcaacaagcgacacaaagcccgg

SEQ ID NO: 22 - DNA, 93 bp, Artificial
  13F2
caactgtaatacgactcactataggagagaaggaaagagagaaaaaaaaaacgcaaagcgccgccatcggatgccaagcgacacaaagcccgg

SEQ ID NO: 23 - DNA, 94 bp, Artificial
  15F1
caactgtaatacgactcactataggagagaaggagggaagagagaaaaaatcgacggaacaggggccccctgtggaaagcgacacaaagcccgg

SEQ ID NO: 24 - DNA, 95 bp, Artificial
  15F3
caactgtaatacgactcactataggagagaaggagggaagagagaaaaaaaacatagggaacccaggtgattggggcaagcgacacaaagcccgg

SEQ ID NO: 25 - DNA, 71 bp, Artificial
  17F3*
caactgtaatacgactcactataggagactgtcccaacggcaatgtggctcacaagcgacacaaagcccgg

SEQ ID NO: 26 - DNA, 71 bp, Artificial
  18F4*
caactgtaatacgactcactataggagacaggattgacaccgaccacttgcccaagcgacacaaagcccgg

SEQ ID NO: 27 - RNA, 26 nt, Artificial
  15F1 Clone
ucgacggaacaggggcccccugugga

SEQ ID NO: 28 - RNA, 26 nt, Artificial
  15F1-M1 Clone
acgacggaacaggggcccccugugga

SEQ ID NO: 29 - RNA, 26 nt, Artificial
  15F1-M2 Clone
ucgacggaaccggggcccccggugga

SEQ ID NO: 30 - RNA, 26 nt, Artificial
  15F1-M3 Clone
ucgacggagcaggggcccccugcgga

SEQ ID NO: 31 - RNA, 25 nt, Artificial
  15F3 Clone
cauagggaacccaggugauuggggc

SEQ ID NO: 32 - RNA, 25 nt, Artificial
  15F3-M1 Clone
cagagggaacccaggugauuggggc

SEQ ID NO: 33 - RNA, 25 nt, Artificial
  15F3-M2 Clone
cauagggaacccaggagauuggggc

SEQ ID NO: 34 - RNA, 25 nt, Artificial
  15F3-M3 Clone
cauagggaacccaggugacuggggc

SEQ ID NO: 35 - RNA, 25 nt, Artificial
  15F3-M4 Clone
cauagggaacgccggugauggcggc

SEQ ID NO: 36 - DNA, 21 bp, Artificial
  Pre Optimized Capture Sequence
ggaggaagaggaagggagagg

SEQ ID NO: 37 - DNA, 9 bp, Artificial
  Pre Optimized Spacer Sequence
gagaggaag

SEQ ID NO: 38 - DNA, 21 bp, Artificial
  Post Optimized Capture Sequence
ggagagaaggagggaagagag

SEQ ID NO: 39 - DNA, 10 bp, Artificial
  Post Optimized Spacer Sequence
aaaaaaaaaa

SEQ ID NO: 40 - RNA, 50 nt, Artificial
  15F1-parent
aaaaaaucgacggaacaggggcccccuguggaaagcgacacaaagcccgg

SEQ ID NO: 41 - RNA, 50 nt, Artificial
  Mut1 (2-1A)
aaaaaaacgacggaacaggggcccccuguggaaagcgacacaaagcccgg

SEQ ID NO: 42 - RNA, 50 nt, Artificial
  Mut2 (2-2G)
aaaaaaucgacggaaccggggcccccgguggaaagcgacacaaagcccgg

SEQ ID NO: 43 - RNA, 50 nt, Artificial
  Mut3 (2-3C)
aaaaaaucgacggagcaggggcccccugcggaaagcgacacaaagcccgg

SEQ ID NO: 44 - RNA, 51 nt, Artificial
  15F3-parent
aaaaaaaacauagggaacccaggugauuggggcaagcgacacaaagcccgg

SEQ ID NO: 45 - RNA, 51 nt, Artificial
  Mut1 (3-1G)
aaaaaaaacagagggaacccaggugauuggggcaagcgacacaaagcccgg

SEQ ID NO: 46 - RNA, 51 nt, Artificial
  Mut2 (3-2A)
aaaaaaaacauagggaacccaggagauuggggcaagcgacacaaagcccgg

SEQ ID NO: 47 - RNA, 51 nt, Artificial
  Mut3 (3-3C)
aaaaaaaacauagggaacccaggugacuggggcaagcgacacaaagcccgg

SEQ ID NO: 48 - RNA, 51 nt, Artificial
  Mut4 (3-4G)
aaaaaaaacauagggaacgccggugauggcggcaagcgacacaaagcccgg

SEQ ID NO: 49 - RNA, 48 nt, Artificial
  Mut1-Long stem
aaaaaaaaaagcgcccaggugauugggcgcaagcgacacaaagcccgg